(19) United States
(12) Patent Application Publication
(10) Pub. No.: US 2012/0310105 A1
(43) Pub. Date: Dec. 6, 2012

(54) MONITORING TASK ENGAGEMENT USING BETA OSCILLATIONS

(75) Inventors: Joseph Feingold, Chestnut Hill, MA (US); Daniel J. Gibson, Cambridge, MA (US); Ann M. Graybiel, Lincoln, MA (US)

(73) Assignee: MASSACHUSETTS INSTITUTE OF TECHNOLOGY, Cambridge, MA (US)

(21) Appl. No.: 13/485,540

(22) Filed: May 31, 2012

Related U.S. Application Data
(60) Provisional application No. 61/491,656, filed on May 31, 2011.
(60) Provisional application No. 61/501,047, filed on Jun. 24, 2011.

(51) Int. Cl. A61B 5/048

(52) U.S. Cl. 600/544

(57) ABSTRACT

The invention provides methods and apparatuses for monitoring task engagement by measuring beta frequency oscillations in the brain. Changes in power, frequency, and/or coherency of beta frequency oscillations correlate to an individual's engagement in or disengagement from a task. Accordingly, beta frequency oscillations can be used to determine if an individual prematurely disengages from a task, to diagnose if a person has a disorder affecting task engagement, and to evaluate the efficacy of a treatment for a disorder.

### REFERENCE TO RELATED APPLICATIONS

This application claims priority to and the benefit of U.S. Provisional Application No. 61/491,656, filed on May 31, 2011, and U.S. Provisional Application No. 61/541,047, filed on Jun. 24, 2011, the entire disclosures of each of which are incorporated by reference herein.

### STATEMENT REGARDING FEDERALLY FUNDED RESEARCH

This invention was supported by grants N00014-07-1-0903 awarded by Office of Naval Research (ONR), NBCHC070105 awarded by Defense Advanced Research Projects Agency (DARPA), and R01NS025529 awarded by National Institute of Neurological Disorders and Stroke (NINDS). The government has certain rights in the invention.

### BACKGROUND

Oscillations in the beta frequency band (13-30 Hz) range in sensorimotor cortex have long been associated with movement. The hallmark of beta activity in cortical motor areas is a pattern of peri-movement beta suppression (relative to the resting state) followed by an increase in beta activity post-movement. This pattern has been observed in local field potential (LFP) and EEG recordings from cortical motor areas in humans and monkeys performing simple single or repetitive movements, such as finger presses or wrist movements in response to a cue. Typically, beta activity fell to a minimum following cue presentation or the start of movement execution, and reached a maximum immediately following movement.

In view of this pattern, beta was labeled an “idling” frequency, with beta band oscillations theoretically representing a minimal energy state that the brain enters in the absence of processing. However, the finding of pathologically excessive beta band synchrony in patients with Parkinson's disease (PD) and animal models of Parkinsonism, led to the idea that beta might be more specifically anti-kinetic or anti-movement in nature. Rather than representing idling, this theory suggests that beta oscillations (or the mechanisms that give rise to them) actively determine if movement is to occur. The inverse correlation between beta activity and movement has been used to account for the increase in beta activity observed during sustained motor output, including the maintenance of a precision grip or the application of a constant force. These findings give further support to the notion of beta activity representing not the absence of motor output, but rather a decreased likelihood of changing the existing motor output. More recently, this connection between beta activity and preserving the status quo was extended to cognitive processes, where the role of beta oscillations in brain regions involved in cognitive processing is to preserve the current state. According to this idea, beta oscillations in a given brain region should be higher when that region does not anticipate an impending change in motor/cognitive output or set, and so maintains its current pattern of activity.

Despite their theoretical appeal, none of these interpretations fits well with the core phenomenon of maximal beta activity occurring in cortical motor areas immediately following movement. If beta activity reflects idling or conservation of the current state or output, why is it highest following movement, but relatively low during rest (behavioral “idling”)?

Excessive beta band (13-30 Hz) activity in cortico-basal ganglia circuits is recognized as a pathophysiological signature of Parkinson's disease. Yet, the function of beta oscillations in the healthy primate brain is not clear. Current theories link beta activity to idling or the preservation of the current state of cortical areas.

### SUMMARY

The application is based in part on the discovery that beta oscillations in the local field potentials (LFPs) in frontal cortex and striatum of monkeys performing arm movements are not directly related to movement, but to the completion of behavioral performance, and the disengagement of the brain region from direct involvement in that behavior. Beta oscillations occurred in brief, spatially localized bursts that were most pronounced following movement or task performance. Beta bursts were detected based on their relative contribution to the spectrum of the LFP signal, as opposed to their overall amplitude. The rates and power of beta bursts differed across brain regions studied. Post-performance beta burst rates and power tracked the details of the preceding task performance differently by brain region. Moreover, in striatum and prefrontal cortex, beta burst rates were higher following correct trials than errors, and the bursts at pairs of sites across these regions were coherent. Beta oscillations represent post-performance reinforcement of the network activity that led to the desired behavioral outcome obtained immediately prior to the post performance beta activity.

Moreover, the timing of beta bursts is substantially random during task performance, and become significantly more coherent (e.g., coordinated) across brain regions after task performance. Relative measurement of the timing of bursts therefore can be used to monitor task engagement in real time. In addition, burst rate can be used to monitor task engagement in real time. Because beta oscillations correlate to proper task completion, beta oscillations can be used to monitor an individual's engagement in or premature disengagement from a task. Similarly, beta oscillations can be used to diagnose learning disabilities where an individual fails to engage or to stay engaged in a cognitive task. The methods disclosed herein also can be used to diagnose cognitive conditions, such as but not limited to Parkinson's Disease, that are typified by abnormal beta oscillations (e.g., hyperactivity or hypoactivity).

The invention provides, in part, a method of assessing whether an individual is engaged in a task. The method includes the steps of measuring an EEG, for a plurality of frequencies, of the individual prior to, during and after task performance using a plurality of electrodes to generate a data set comprising frequencies and power; selecting, using a computer, frequencies in the beta frequency range from the EEG data set; comparing, using a computer, the power in the frequencies in the beta frequency range prior to, during and after task performance; and determining whether the person was engaged in the task in response to the comparison of the power in the beta frequency range prior to, during, and after task performance.

The invention also provides, in part, a method of assessing whether an individual is engaged in a task. The method includes the steps of measuring an EEG, for a plurality of frequencies, of the individual prior to, during and after task performance using a plurality of electrodes to generate a data set comprising frequencies and burst rate; selecting, using a computer, frequencies in the beta frequency range from the EEG data set; comparing, using a computer, the burst rate in the frequencies in the beta frequency range prior to, during and after task performance; and determining whether the person was engaged in the task in response to the comparison of the burst rate in the beta frequency range prior to, during, and after task performance.

The invention also provides, in part, a method of assessing whether a brain region is engaged in a task, the method comprising: monitoring, using a computer, brain activity in the beta frequency range prior to, during, and after performance of a task, wherein a burst in beta frequency activity after performance of the task is indicative that the brain region has disengaged from performance of the task.

Embodiments of the methods can include one or more of the following features:

The data set can be a sample obtained from the patient.

The comparison is made by plotting frequency and power against time.

The measurement of task engagement is made for a plurality of brain regions.

The method includes the additional step of measuring coherence in the beta frequency range between brain regions of the plurality of brain regions.

The comparison is performed by a computer by transforming the data. For example, the transformation can be performed by a filter. The transformation can be a HHT transform and/or a FFT transform.

The individual is considered to have been engaged in the task if beta frequencies prior to task performance during resting have decreased power relative to the beta frequencies after task performance.

The beta frequency range is about 13 Hz to about 30 Hz.

The brain region is selected from the group consisting of primary motor and dorsal premotor cortex, dorsolateral prefrontal cortex, caudate nucleus, and putamen.

The invention also provides, in part, a method of diagnosing a condition affecting movement or thought preparation and movement cessation in an individual. The method can include measuring, using an electroencephalographic computer, brain activity in the beta frequency range prior to, during, and post performance of a task, wherein a burst in beta frequency activity during the task completion time in a normal brain region is indicative that the brain region has prematurely disengaged from performance of the task.

In some embodiments, the method can include the step of measuring beta frequency activity in primary motor and dorsal premotor cortex, dorsolateral prefrontal cortex, caudate nucleus, and putamen.

The invention also provides, in part, a method of diagnosing a condition affecting movement or thought preparation and cessation in an individual. The method can include measuring, using a computer, brain activity in the beta frequency range before, during and after performance of a task, wherein bursts in beta frequency activity that are not coherent with bursts in other brain regions, as compared to normal brains, are indicative that brain region activity is abnormally coordinated.

In some embodiments, the method can include the step of measuring the coherence of beta frequency activity in at least two brain regions.

In some embodiments, the at least two brain regions comprise striatum and prefrontal cortex.

In some embodiments, the at least two brain regions comprise caudate nucleus and dorsolateral prefrontal cortex.

The invention also provides, in part, an apparatus for assessing whether an individual is engaged in a task. The apparatus includes: a plurality of electrodes for placement on the head of the individual; and a computer system. The computer system includes (e.g., on one or more suitably programmed mediums) an EEG module, in communication with the plurality of electrodes, for measuring an EEG, for a plurality of frequencies, of the individual prior, during and post task using a plurality of electrodes to generate a data set of frequencies and power; a selection module in communication with the EEG module for selecting frequencies in the beta frequency range from the EEG data set; a comparator for comparing the power in the frequencies in the beta frequency range before, during and post task. The system also can include a display, in electrical communication with the comparator, for showing whether the person was engaged in the task, in response the comparison of the power in the beta frequency range before, during, and post task.

The invention also provides, in part, a method of assessing the efficacy of a drug used to treat an individual with a cognitive disability typified by hyperactive beta oscillations. The method can include the steps of measuring an EEG at a first time, for a plurality of frequencies, of the individual prior to, during and after task performance using a plurality of electrodes to generate a first data set comprising frequencies and power, the first time being prior to administration of the drug; measuring an EEG at a second time, for a plurality of frequencies, of the individual prior to, during and after task performance using a plurality of electrodes to generate a second data set comprising frequencies and power, the second time being after administration of the drug; selecting, using a computer, frequencies in the beta frequency range from the first EEG data set and the second EEG data set; comparing, using a computer, the power in the frequencies in the beta frequency range prior to, during and after task performance in the first EEG data set and in the second EEG data set; and determining whether administration of the drug decreased beta frequency oscillations.

In some embodiments, cognitive disability is selected from the group consisting of: Parkinson's Disease, obsessive compulsive disorder, autism, attention deficit disorder, attention deficit hyperactivity disorder, and post traumatic stress disorder.

### BRIEF DESCRIPTION OF DRAWINGS

The figures are not necessarily to scale, emphasis instead generally being placed upon illustrative principles. The figures are to be considered illustrative in all aspects and are not intended to limit the invention, the scope of which is defined only by the claims.

FIG. 1 is an experimental design of a monkey playing a video game analogue.

FIG. 2 is an experimental design of behavioral tasks: Single Movement (Sing) task, Sequentially (Seq) and Simultaneously (Sim) Cued Sequential Movement tasks, Simultaneously Cued Single Movement (SimSing) task, and NoGo task.

FIG. 3 is a graph showing beta oscillations in the caudate nucleus before, during, and after a behavioral task.

FIGS. 4A-B. FIG. 4A is the same as FIG. 3. FIG. 4B shows the envelope of the beta (14-22 Hz) HHT composite for each of the ˜100 individual trials whose bandpass filtered power was averaged to create the spectrogram of FIG. 3, indicating that beta oscillations occur in bursts.

FIG. 5 shows beta bursts in LFPs simultaneously recorded in dorsolateral prefrontal cortex (dlPFC) and caudate nucleus (CN) in a single trial.

FIGS. 6A-B show beta bursts in dlPFC from one monkey (A) during a portion of the inter-trial interval recorded between correct trials of the Sing task and (B) during the entire trial.

FIG. 7 shows modulation of beta-band power in dlPFC in NoGo and Sim tasks.

FIG. 8 shows a HHT-based reconstruction of the beta-band content of an LFP signal from dlPFC (A) during a portion of the pre-reward interval recorded in a correct trial of the Sing task and (B) during the entire trial.

FIG. 9 shows beta bursts in CN from one monkey (A) as in FIG. 8A, for the ITI and (B) as in FIG. 8B.

FIG. 10 shows the trial-averaged rate of beta bursts for each LFP (pooled across multiple recording sessions from two monkeys) recorded in dlPFC and CN across tasks.

FIG. 11 is an average coherogram across all pairs of simultaneously recorded LFPs in contralateral CN and dlPFC across ˜100 correct trials of the simultaneously cued (Sim) sequential arm movement task.

FIGS. 12A-B show (A) experimental designs of behavioral tasks and (B) suppression-rebound pattern of the task-modulation of beta power across simultaneously recorded LFPs in M1PMC, dlPFC, CN and Put.

FIG. 13 shows the beta power in each of four brain regions exhibited a pattern of peri-cue suppression, followed by a rebound during the post-trial period.

FIGS. 14A-C show spatially localized bursts of beta oscillations in M1PMC, dlPFC, CN and Put. (A) An individual trial; (B) a comparison of beta bursts across the population of simultaneously recorded sites; and (C) peak cross-covariance between the envelopes of beta bursts recorded at pairs of sites.

FIG. 15 shows a comparison of beta bursts across the population of simultaneously recorded sites across the four brain regions.

FIGS. 16A-E show differential modulation of population beta burst rates in M1PMC, dlPFC, CN and Put by behavioral tasks (A) as a time-course of the trial-averaged beta band power and (B-E) as a function of the preceding behavioral task.

FIG. 17 shows phase of the coherence between bursts in the LFPs from the closest pair of simultaneously recorded electrodes in the CN.

FIGS. 18A-D show beta range coherence between dlPFC and CN is highest in the post-trial period and disproportionately due to bursts. (A,C) Population average coherence between all pairs of simultaneously recorded LFPs in the CN and dlPFC. (B,D) Post-trial coherence in CN and dlPFC.

FIGS. 19A-F shows (A) population average burst power (normalized to rest) across task epochs; (B) absolute burst rates; and (C-F) LFPs in each brain region.

FIGS. 20A-H summarize the various measures that can be used to detect an individual's (and individual brain region's) engagement in task performance, and the point at which the performance is (correctly) completed.

### DETAILED DESCRIPTION

Beta oscillations in the local field potentials (LFPs) in frontal cortex and striatum of monkeys performing arm movements are not directly related to movement, but to the offset of behavioral performance. In all brain regions, beta oscillations occurred in brief, spatially localized bursts that were most pronounced following movement or task performance. Beta bursts were detected based on their relative contribution to the spectrum of the LFP signal, as opposed to their overall amplitude. The rates and power of beta bursts differed across brain regions. Post-performance beta burst rates and power tracked the details of the preceding task performance differently by brain region. Moreover, in striatum and prefrontal cortex, beta burst rates were higher following correct trials than errors, and the bursts at pairs of sites across these regions were coherent. Based on our results, beta oscillations represent post-performance reinforcement of the network activity that led to the desired behavioral outcome, movement or result, obtained immediately prior to the beta activity.

Prominent post-movement increases in beta power may be a ubiquitous signature of post-performance processing in the brain. This hypothesis was tested in monkeys by recording LFPs in cortico-striatal regions known to be directly involved in motor control, movement sequencing and executive function. We found that LFPs in frontal cortex and striatum were characterized by brief, spatially localized episodes, during which beta frequencies disproportionately dominated the LFP spectrum. These beta bursts were most pronounced following behavioral performance, when they were modulated by specific features of the preceding behavior, as well as the outcome of that behavior. Based on our results, we propose that the role of beta oscillatory activity might not be to preserve the current state or behavioral output of individual brain regions, but to preserve or reinforce the cortico-striatal network dynamics that led to the desired outcome obtained immediately prior.

Based on human electroencephalogram (EEG) and magnetoencephalograpy (MEG), and based on monkey local field potential (LFP) experiments, using simple single movement tasks—such as wrist flexion and precision grip—activity in the beta band (roughly 13-30 Hz, depending on the species, subject and brain region) has been linked to brain idling. Studying visually cued single and sequential arm movements, as well as rest periods, it was discovered that across regions of the frontal cortex and striatum of monkeys, beta activity changes on a considerably faster time-scale than previously thought, appearing in brief (˜150 ms) bursts of oscillations, during which beta-band power dominates the spectrum. These changes in the EEG or LFP can be detected with high temporal resolution using a custom-modified version of the Hilbert-Huang transform (HHT), making it possible to track beta activity in response to individual behavioral events, without averaging the results over multiple trials. It is hypothesized that these beta bursts represent packets of communication or local processing. The timing of these bursts in a given brain region appears to correlate with the end of the brain's involvement in behavior. During these times, increased coherence was detected in the beta band between frontal cortex and striatum. Based on these findings, it is hypothesized that, in contrast to the idling interpretation, beta activity represents packets of communication or local processing, which become prominent when a brain region disengages from behavior. Beta bursts might thus function as “offline” data dumps and/or can facilitate periods of network tuning/updating following a brain region's engagement in a task.

Thus, bursts in the beta frequency band of signals from the human or animal brain can be used to detect the precise temporal boundaries of individual brain regions' engagement in a given task, as well as the depth (or level) of that engagement. As a result, the present invention can be used for a number of applications including, by way of non-limiting example:
1. Neurophysiological measures of learning and performance: improvement and monitoring of brain-site-specific performance of cognitive or motor tasks across repeated trials.
2. Continuous performance monitoring: detection of lapses in task engagement (e.g., for collision avoidance, operating room safety, etc.).
3. Diagnostic Tools: detection of the neural signature of task completion and the depth of engagement in the task (e.g., for OCD, ADD, PTSD and learning disabilities), as well as the coherence between bursts across brain regions during post-engagement (as a measure of network updating).
4. Basis for targeted therapies: brain-site-specific therapeutic interventions for treating task-engagement deficiencies, based on burst detection and analysis.

In order to study beta burst phenomena, analytical methods were developed that are based on the Hilbert transform, rather than the Fourier transform. This new approach is based largely on recent advances in the application of the Hilbert transform to nonlinear data, known as the Hilbert-Huang transform (HHT) (Huang et al., 1998 & 2003). Using a process termed Emprical Mode Decomposition (EMD), the HHT constructs Intrinsic Mode Functions (IMFs) that are AM-FM analogues of Fourier components of the Fourier transform. In contrast to the potentially infinite number of Fourier components needed to recreate a waveform, the IMFs capture the intrinsic timescales of the raw data in compact form, enabling faithful reconstruction of the raw signal with only a handful of IMFs.

Among the strengths of the HHT is its definition of a meaningful instantaneous frequency, which gives rise to the possibility of analyzing the short bursts of beta activity that we have observed in the raw LFPs. The HHT makes it possible detect the timing, duration, frequency and amplitude of bursts much more accurately than can be done with traditional Fourier-based methods. One embodiment of the HHT was implemented using the Matlab (Mathworks, Natick, Mass.) software package, in order to construct IMFs from raw brain signals. A custom-made algorithm was developed to detect consistent results obtained from the HHT, by performing the EMD repeatedly with different parameter values and defining confidence limits. The entire processing pipeline was performed from end to end, including making composites, for each iteration of parameter values. We defined the CLs as +/−2*SEM away from the mean over all iterations. The mean over iterations then became our composite HHT waveform for subsequent analysis.

A separate algorithm was created for determining the contribution of the IMFs to the content of individual frequency bands, by summing IMFs, appropriately weighted by frequency, in order to construct an HHT composite (an analogue of the band-pass filtered signal). “Appropriately weighted by frequency” means weighted by

exp(−(f−mu)̂pow)/(2*sigmâpow))

where mu=mean(freqlim) and sigma=mu−freqlim(1).

Finally, an algorithm was developed for detecting the boundaries of bursts in the HHT composite, by assessing at each time point the goodness-of-fit of the HHT composite to the LFP, and imposing frequency and phase constraints on the HHT composite, as well as requiring it to behave in sinusoidal-like fashion (e.g., a zero-crossing must exist between successive extrema). The algorithm is described as follows:
1. Find local maxima in the composite envelope (or in the bandpass-filtered envelope);
2. Searching outwards from each local maximum, find the closest points on each side where the absolute difference between the composite HHT and the original waveform exceeds a threshold;
3. Again searching outwards, search the interval where the absolute difference does not exceed threshold for “bad extrema”, i.e. composite HHT waveform maxima that have negative values or minima that have positive values, and trim the search interval to exclude them;
4. Again searching outwards within the trimmed search interval, search for half-cycles of the composite HHT over which its phase departs by more than a quarter-cycle from the phase expected for a constant frequency oscillation, and trim the search interval again to exclude them;
5. Searching INWARDS from the start and end of the trimmed interval of step 3, find the first and last zero crossings and designate them as the start and end of the burst respectively.
6. If the burst no longer exists, eliminate it from the list of bursts; “no longer exists” means either the end is not after the beginning, or the original burst peak is not between the beginning and end, or the burst does not contain at least one full cycle of oscillation.

The discovery of spatially localized, brief, discrete bursts of beta-frequency oscillations in the LFPs recorded from sites across frontal cortex and striatum led us to interpret the trial-averaged beta-band power as indicating the probability distribution of observing a burst of beta oscillations dominating the spectrum of the LFP at each time-point in a given trial. In all brain areas, beta bursts were suppressed (in terms of amplitude and rate of occurrence, relative to rest period levels) during the initial cue presentation period, prior to the monkey's initiation of arm movement. In sequential movement tasks, bursting activity tended to resume during the inter-movement hold periods (or inter trial intervals (ITI)), particularly when these were long (1.4 s), as opposed to short (0.7 s). This pattern of peri-movement burst suppression along with increased bursting activity during relatively long breaks between sequential movements suggests that the modulation of beta bursts can be used to track engagement in task performance. This idea is further supported by an analysis of the timing of peak bursting activity during task performance. In all brain regions, the peak rates and peak amplitudes of beta bursts were significantly higher than the average values during periods of rest. Whereas beta bursts in cortical motor areas occurred most frequently and were highest in amplitude immediately following the last movement in a trial, the rate and amplitude of beta bursts in dorsolateral prefrontal cortex (dlPFC) and striatum peaked in the period immediately following the trial. Given the presumed roles of these different brain regions in task performance, we hypothesize that the beta bursts in a given brain region increase in amplitude and rate after that brain region disengages from task performance.

The modulation of beta activity in the absence of movement also was studied, under two different conditions: first, during periods of rest, in which the monkey sat quietly for several minutes; and second, during trials in which visual cues that would normally instruct upcoming arm movements were presented in a different context, so that the monkey scanned the visual cues, but did not respond with any arm movement. It was found that the time-course of beta activity in the latter case differed from the beta activity during rest periods. In contrast to previous reports, the changes in beta in response to the visual cues in the absence of subsequent arm movement, and following reward delivery were similar to those during the movement tasks. Thus, these changes in beta activity correspond to periods of the brain's engagement with external stimuli and reward, representing cognitive processing regardless of motor output. Collectively, the changes in beta activity signal the engagement (low beta activity relative to rest) and subsequent disengagement (high beta activity relative to rest) from behavioral task performance, regardless of motor output.

It has been further found that across brain sites, there is a strong, positive correlation between the average rate of post-engagement beta bursts and the number of movements or visual cues in the task. This finding suggests that changes in beta bursts not only signal the end of an engagement in a task, as opposed to ongoing periods of rest, but are directly related to the cognitive load during task performance; the more demanding the task, the greater the number of bursts following task performance.

In support of this conclusion, two tasks were compared in which the monkey performed sequences of three arm movements each. The monkey performed identical movement sequences in both tasks. The tasks differed only with respect to the timing of the presentation of the instructional cues. In the first task, the cues instructing the entire sequence were presented simultaneously at trial start and remained unchanged throughout the trial. In the second, only the cue instructing the first movement appeared at trial start, with each successive cue (instructing the next movement in the sequence) appearing at the offset of the preceding movement. We found that the burst rates during the trial were higher in the simultaneously cued task than the sequentially cued task. This pattern was reversed in the interval immediately following the trial. In those trials in which the cues instructing subsequent arm movements were presented in advance of the movements, the brain regions in question beta-burst relatively more than they did during trials in which the cues instructing upcoming movements were not presented ahead of time. Remarkably, in the period immediately following the trials (when the brain regions presumably disengaged from task performance), there were more beta bursts in the sequentially cued task than in the simultaneously cued task.

This pattern of results suggests that the brain regions beta-burst opportunistically, depending on the information available to them and the ongoing demands of the task. If a region can disengage briefly from task performance, then it will take the opportunity to do so, as indicated by the occurrence of beta bursts. However, the fact that the brain regions switch from bursting more during the trial, when the cues are presented ahead of time, to bursting more following the trial, when the cues are not presented ahead of time, argues against the idea that, when the brain regions disengage, they are simply idling. Rather, the beta bursts that occur as a brain region disengages from task performance appear to be involved in the processing of that performance, potentially integrating the cues, movements and outcome, and coordinating these results across brain sites. This proposed function of beta bursts would be relevant to adaptive learning in a dynamic environment, in which the brain must monitor and optimize the relationship between stimuli and responses in order to maximize its chances of obtaining desired outcomes in the future.

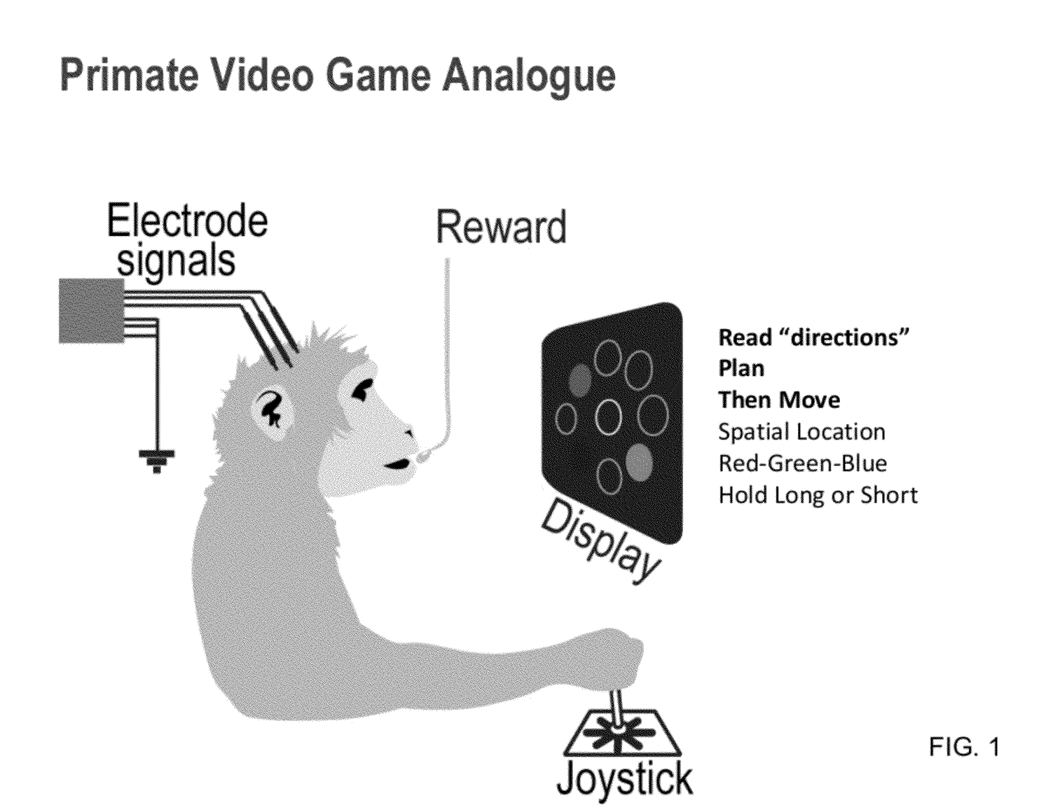

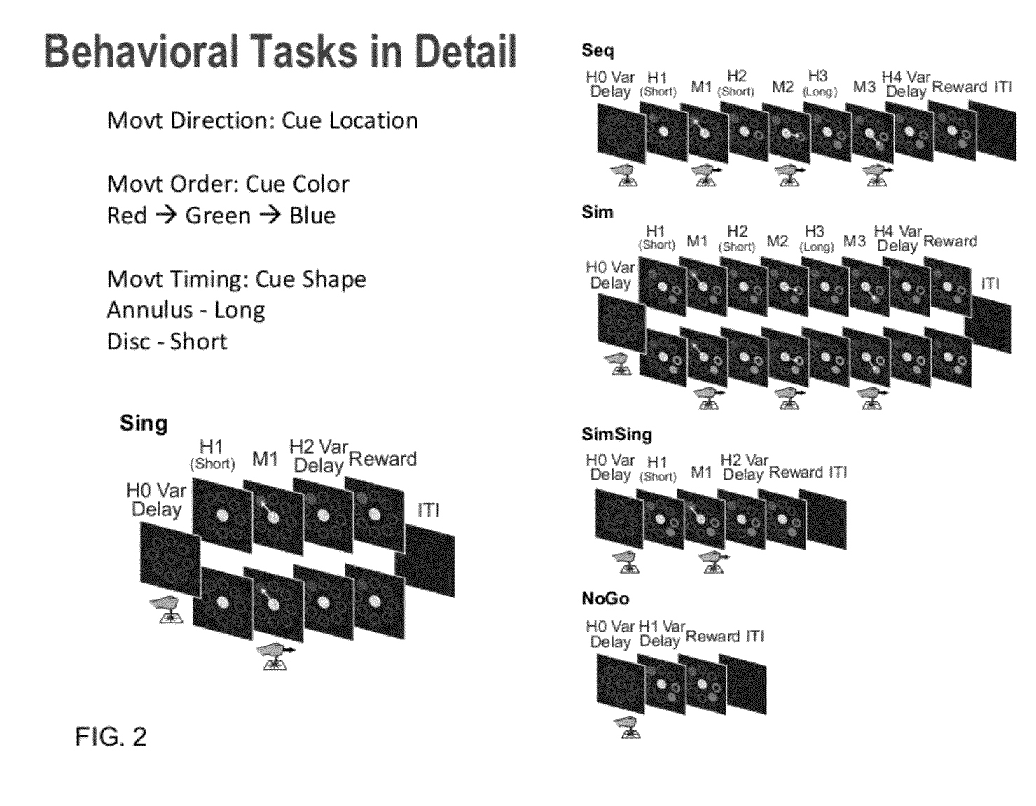

FIG. 1 is an experimental design of a monkey playing a video game analogue. At the start and end of each recording session, data were collected during rest periods with a head-fixed monkey sitting quietly inside or outside of a recording booth. In each recording session, the monkey performed blocks of trials of the behavioral tasks shown in FIG. 2. Experimental trials were run serially, and the time between trials is the inter-trial interval (ITI). At the start of each trial, a monkey was shown visual cues. The monkey then performed tasks (moving a joystick, or in some cases withholding movement) in response to the visual cues. Upon correct completion of each trial, the monkey stopped moving and was given a food reward of constant amount across trials of all tasks, immediately after which it rested for several seconds during the ITI. At the end of the ITI, the next trial began as new cues were presented to the monkey.

FIG. 2 shows examples of trials of the Single Movement (Sing) task, the Sequentially (Seq) and Simultaneously (Sim) Cued Sequential Movement tasks, the Simultaneously Cued Single Movement (SimSing) task, and the NoGo task. Different tasks are presented in separate blocks. Trials start with the appearance of the empty cue array, which persists for a variable period of time (H0), until the colored cues appear. Depending on the task, the monkey must then perform 0, 1 or 3 center-out-center joystick movements (indicated by white arrows), controlling the position of an onscreen cursor. Each movement is preceded by a self-timed center hold interval (H1-3) of short (0.6-1.2 s) or long (1.4-2.0 s) duration. The colors, spatial locations and shapes of the cues instruct the order, direction, and timing of the joystick movements, respectively. For example, in one trial, the movement order was red, green, blue. Alternative color-schemes are used as controls in different blocks of trials of the Sing and Sim tasks. An annulus indicates a long center hold and a solid disc indicates a short center hold, and the location of the annulus or disc corresponds to the direction of movement. In all tasks, following the last movement, the monkey must hold the joystick at the center position for a variable period (H4) until reward is delivered. In the examples shown for the Seq and Sim tasks, the required sequence is H1: short, M1: up-left, H2: short, M2: right, H3: long, M3: down-right.

The Single Movement Task (Sing) requires the monkey to perform a single center-out-center joystick movement in response to a single peripheral cue, following a short or long self-timed hold period (FIG. 2, Sing). After performing the single movement, the monkey must continue to hold the joystick at the center position for pre-reward interval of variable duration (1.2-2.6 s). Each block of the single movement is comprised of 64 trials—two copies each of all possible combinations of spatial movement direction and pre- and post-movement hold interval durations.

The Sequentially Cued Sequential Movement Task (Seq) requires the monkey to perform sequences of 3 joystick movements (M1-3), each preceded by a short or long self-timed center hold period (H1-3). Each movement is cued, in turn, at the start of the preceding self-timed hold interval (FIG. 2, Seq). Conceptually, each Seq trial resembles 3 concatenated trials of the Sing task, with reward being delivered only following the final movement. Although the monkey must accurately self-time the initiation of each of the 3 movements, the monkey has no opportunity to plan ahead, beyond the upcoming movement. The monkey is externally “stepped” through the sequence, with the most recently appearing cue instructing the upcoming movement to be performed.

The Simultaneously Cued Sequential Movement Task (Sim) requires the monkey to perform sequences of 3 movements (FIG. 2, Sim). The sequences are identical to those used in the Seq task, but with the crucial difference that all of the cues for the entire sequence appear simultaneously at trial-start and remain unchanging on the screen for the duration of the trial. Thus, the monkey can collect the visual information it needs to perform the entire arm movement sequence prior to initiating it. While the motor output in Sim and Seq trials is required to be similar, the monkey can plan ahead only in the Sim task. Moreover, the monkey must proceed from one sequential movement to the next without the benefit of suddenly appearing cues, as in the Seq task.

In each block of the Sim or Seq task the monkey must correctly complete 32 trials, each requiring a distinct spatiotemporal sequence of joystick movements. Each sequence can be broken down conceptually into spatial and temporal templates. There are eight possible temporal templates (H1: short, H2: short, H3: short, through H1: long, H2: long, H3: long) and 8×7×6=336 spatial templates. At the start of every recording session, a unique set of 32 sequences to be used that day is constructed according to a prescription that ensures that each of the 8 temporal templates occurs exactly 4 times in every block of trials. Since the monkeys will have been trained extensively on all combinations of spatial and temporal templates prior to the recording phase of the experiment, little learning is expected to occur within any given recording session.

The Simultaneously Cued Single Movement Task (SimSing) is visually identical to the Sim task, but the monkey must perform only the first of the three cued center-out-center joystick movements (FIG. 2, SimSing). In this task, the green and blue (or aqua and orange) cues serve as distractors. In order to receive reward following the single joystick movement, the monkey must continue to hold at the center position for a variable period of time that is substantially longer than the maximum time that it would have been allowed to wait before initiating the next sequential movement in an ordinary Sim trial. By forcing the monkey to wait for an extended period of time, it rules out the possibility that the monkey was preparing to perform the sequence, rather than the single movement alone.

The Simultaneously Cued No Movement Task (NoGo) is also visually identical to the Sim task, but the monkey must withhold all joystick movement in order to receive reward (FIG. 2, NoGo). Following the appearance of the cues, the monkey must continue holding the joystick at the center position for a variable period of time that is substantially greater than what it would have been allowed to wait before initiating the first movement in a visually identical Sim trial.

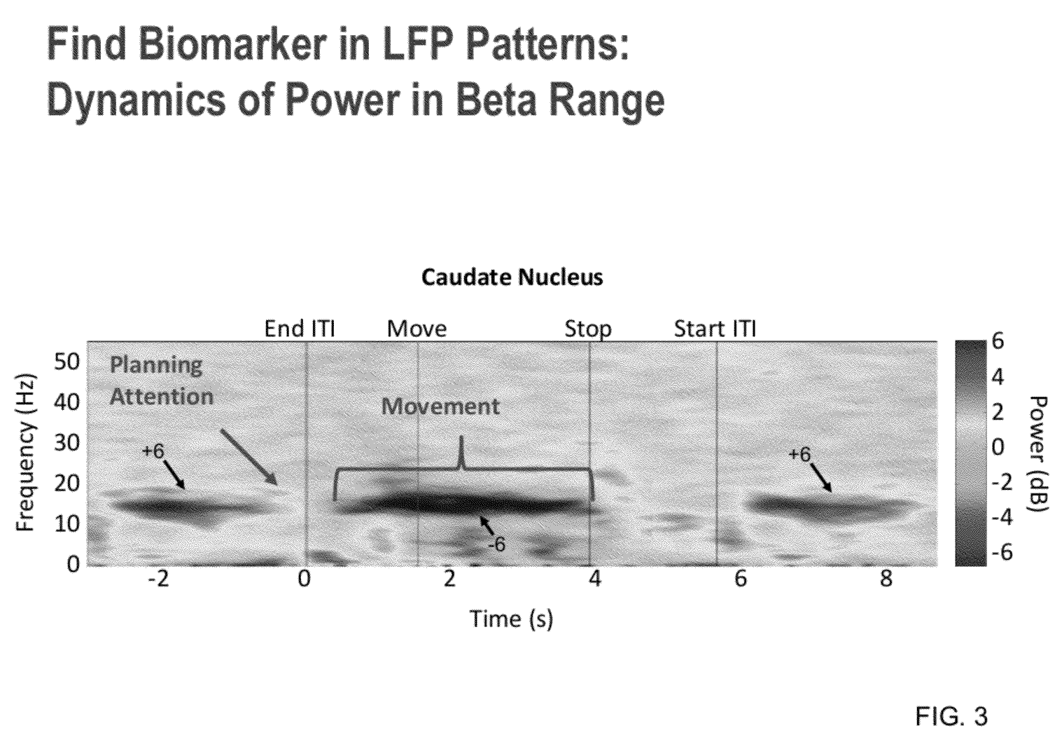

Beta frequency patterns are correlated to task engagement. FIG. 3 is a composite of 10 event-aligned time-windows, showing the power (generated by a multi-taper analysis) averaged across ˜100 correct trials of the Simultaneously Cued task with the Short-Short-Short timing template. The LFPs were recorded from the left caudate nucleus (CN), contralateral to the moving arm. Each of the 10 windows is combined at the median time between successive events. The power in each window is the result of removing the baseline spectrum (to enable visualization by combating 1/f decay typical of LFPs in the brain). The baseline spectrum is the average spectrum across all correct trials for all timing templates of the simultaneously cued task condition (˜864 trials/session). The animal was extremely well practiced at the time these data were taken. Performance was stable across days, as the animal was well acquainted with the task and what it required her to do. Each day different sequences were generated for the trials but, due to the number of sessions and trials, eventually sequences would be reused. End ITI: end of prior inter-trial interval, Move: beginning of joystick movement, Stop: cessation of joystick movement, Start ITI: beginning of next inter-trial interval.

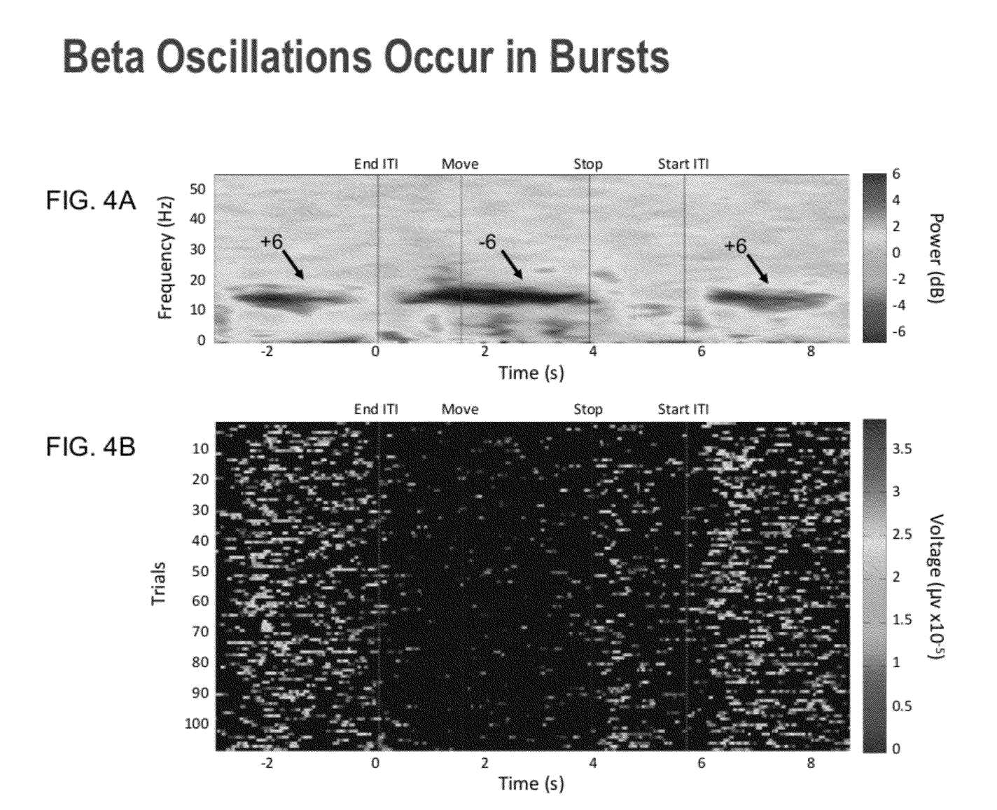

FIG. 4A shows the same spectrogram as FIG. 3 adjacent a HHT composite. FIG. 4B shows the envelope of the beta (14-22 Hz) HHT composite for each of the ˜100 individual trials whose bandpass filtered power was averaged to create the spectrogram of FIG. 3. Our custom-made beta HHT composite is analogous to the beta-band filtered LFP, but with greater temporal resolution, enabling the accurate detection of brief episodes of oscillation, without artifacts of filter-induced ringing. The envelope values are in volts (corresponding to the square-root of power). In contrast to the temporally sustained troughs and peaks in beta activity seen in the movement period and ITI, respectively, of the trial-averaged power (FIG. 4A), FIG. 4B shows that in individual trials, discrete, short-lived bursts of beta power can be detected in the beta HHT composite recorded in the CN.

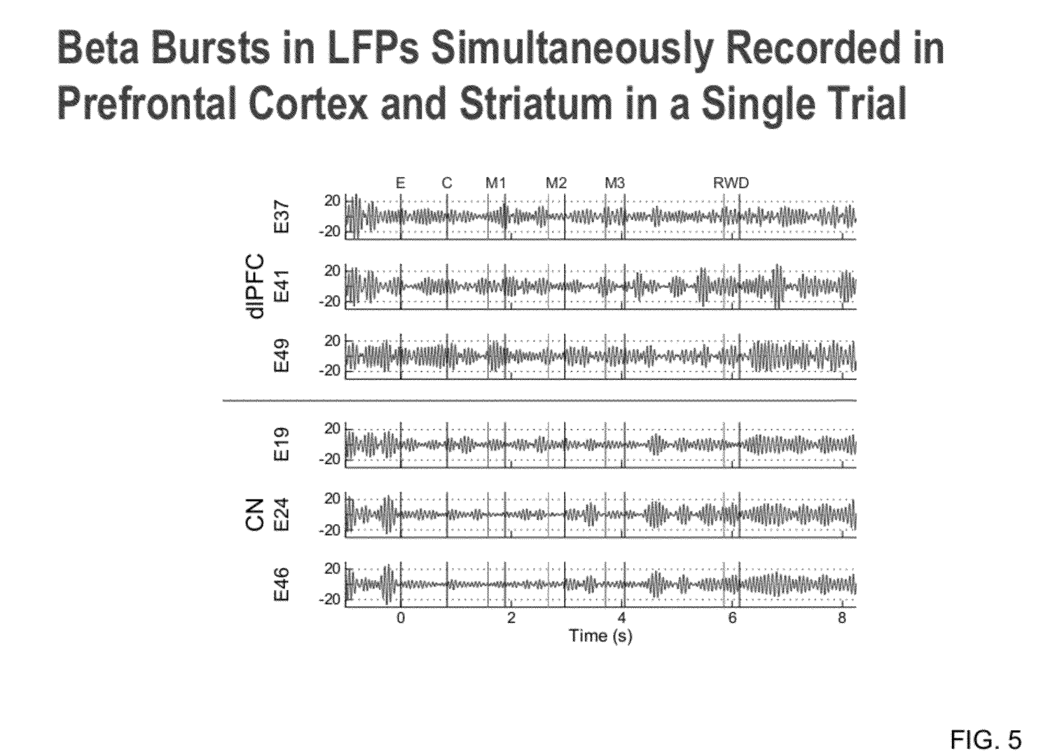

FIG. 5 shows beta bursts in dorsolateral prefrontal cortex (dlPFC) and CN. Beta-band LFPs (filtered for 12-18 Hz) simultaneously recorded at 3 sites in dlPFC (top) and CN (bottom) during performance of a single short-short-short trial of the Sim task. Each trial begins with the onset of an array of empty cues (E). In the Sim task, all of the cues appear simultaneously at the start of the first self-timed center hold interval (C) and remain unchanged on the screen for the duration of the trial, until the ITI (3 sec) begins at the end of the reward delivery (RWD) or once a performance error is detected. The beta-filtered LFPs are characterized by phasic changes in amplitude that last for only a few cycles of beta, and appear throughout the task, but are especially prominent in the ITI.

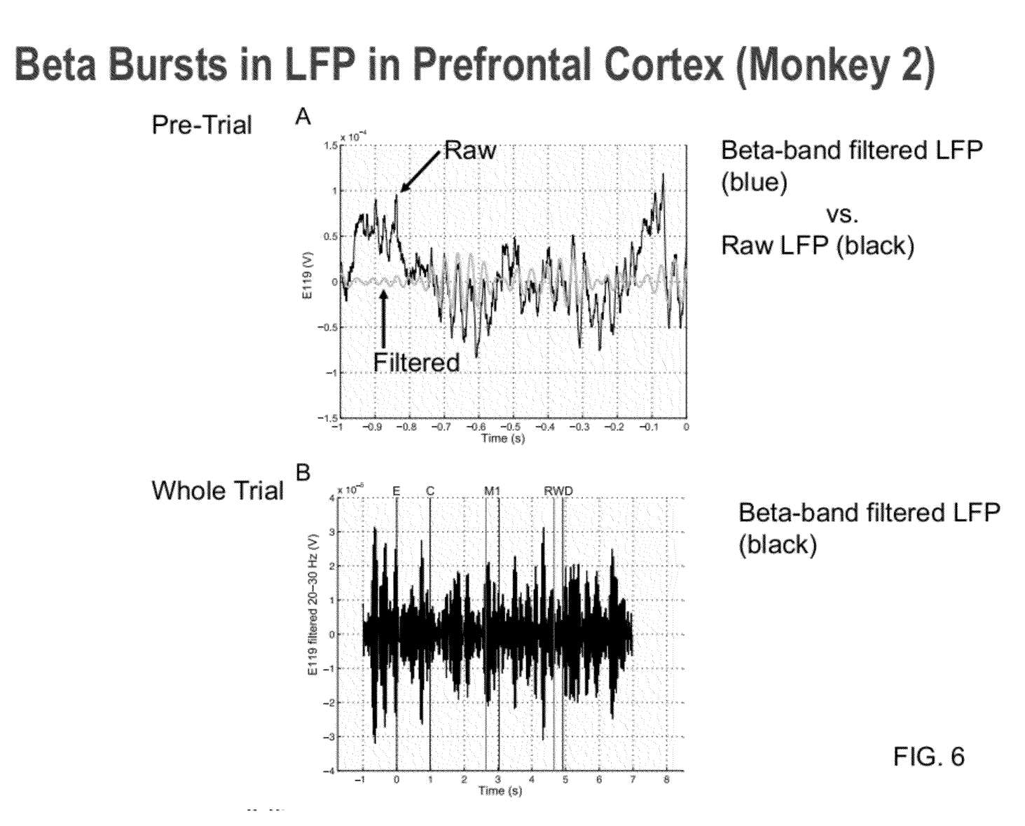

FIG. 6 Beta bursts in dlPFC from one monkey. (A) The 20-30 Hz filtered LFP (blue) is superimposed on the raw LFP signal (black) during a portion of the inter-trial interval recorded between correct trials of the Sing task (empty cue array appeared at time 0). (B) The beta-band filtered LFP, shown for the entire trial, is characterized by brief high-amplitude bursts (task events labeled as in FIG. 5).

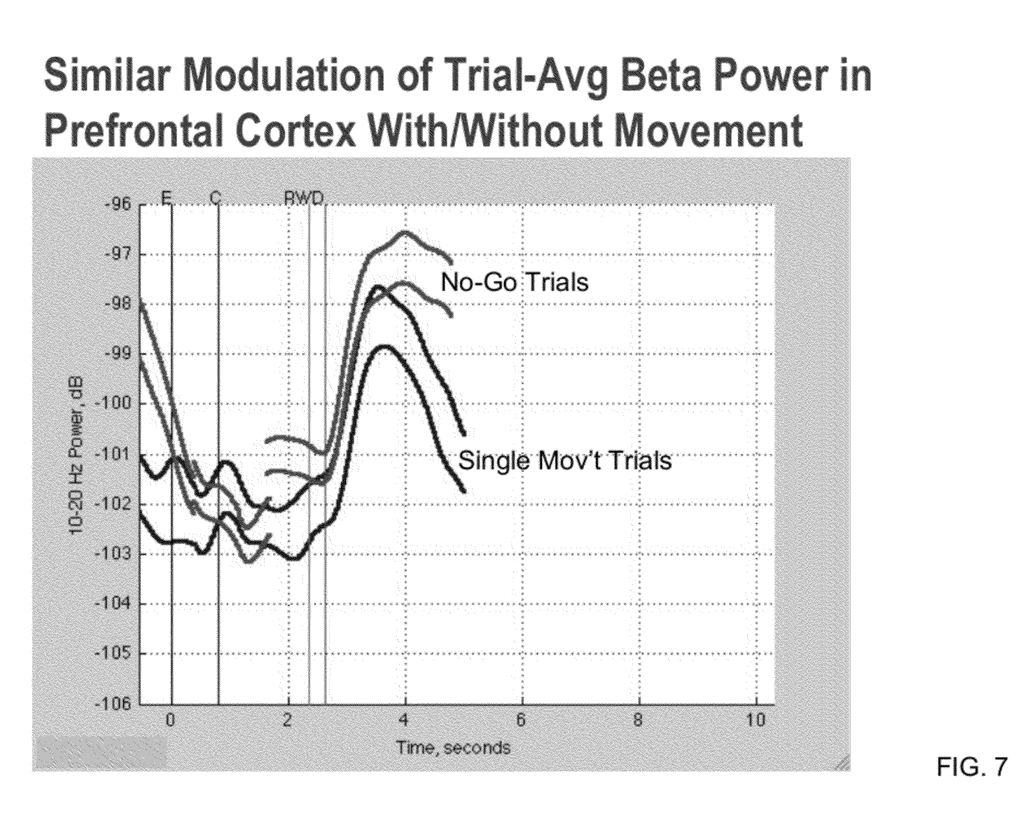

FIG. 7 shows similar modulation of beta-band power in dlPFC in NoGo and Sim tasks. Trial-averaged power (95% confidence intervals) in the beta band from a single electrode in dlPFC is shown for correct trials of the NoGo task (blue) and corresponding epochs of SSS trials of the Sim task (red). The beta power, plotted in 3 windows surrounding the labeled task events, follows a similar pattern of modulation in both tasks, even in the absence of any arm movement.

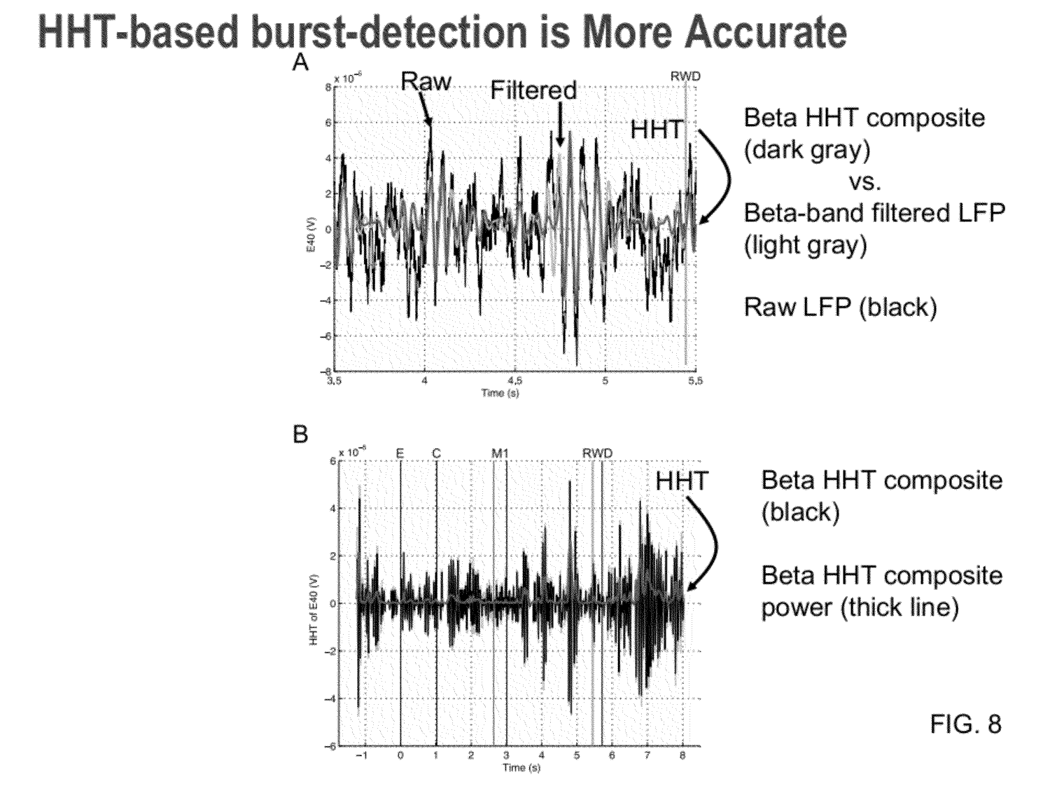

FIG. 8 shows our custom-made HHT-based reconstruction of the beta-band content of an LFP signal from dlPFC. (A) The raw LFP signal (black) is shown along with the 13-20 Hz filtered LFP (blue) and HHT-based reconstruction (red) in a portion of the pre-reward interval recorded in a correct trial of the Sing task (vertical RWD line marks onset of reward delivery). The HHT and fast Fourier transform (FFT)-based methods produce qualitatively similar results, but the former describes individual bursts more accurately than the latter, particularly when the bursts are short. We defined a set of criteria (including goodness-of-fit to the LFP, sinusoidal-like behavior and frequency and phase constraints) for detecting beta bursts in the HHT. Following detection of the temporal boundaries of each burst, we can measure and compute statistics on various burst characteristics, including duration and peak amplitude. (B) The HHT reconstruction of the LFP in the beta range (black) and the power (red) are shown for the entire trial (task events labeled as in FIG. 5).

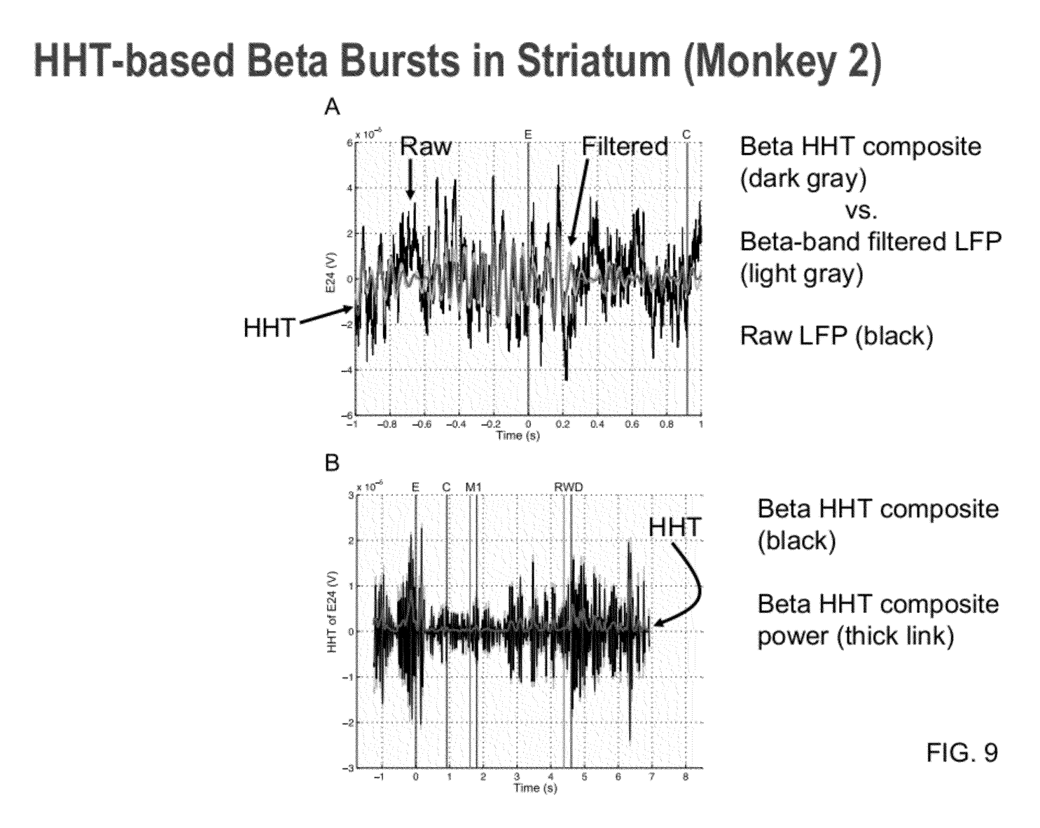

FIG. 9 shows beta bursts in CN from one monkey. (A) As in FIG. 8A, for the ITI. (B) As in FIG. 8B.

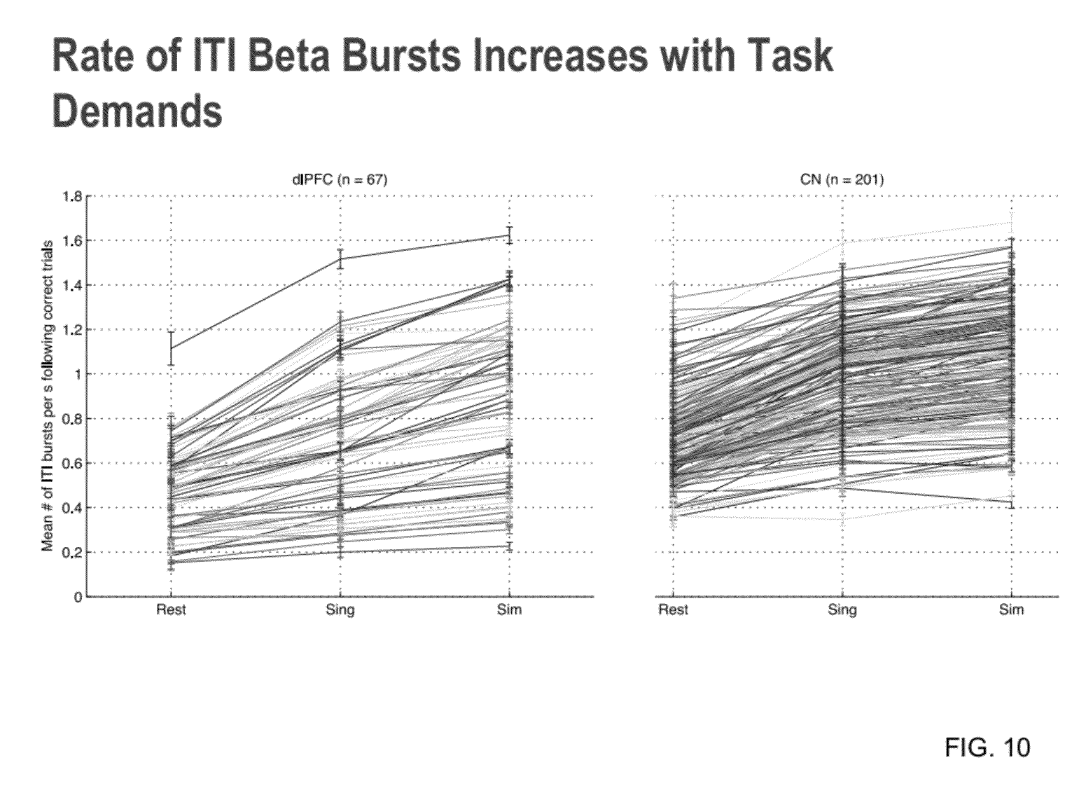

FIG. 10 shows the trial-averaged rate of beta bursts for each LFP (pooled across multiple recording sessions from two monkeys) recorded in dlPFC and CN across tasks. The mean burst rate and 95% confidence limits are plotted in a separate color for each LFP. A majority of LFPs in both brain structures are characterized by burst rates that are significantly elevated (relative to resting periods) during the ITI following correct trials of the behavioral tasks. In addition, the beta burst rates during the ITI are higher following three-movement trials than single-movement trials, suggesting that burst rates are influenced by the demands of the preceding task performed by the monkey.

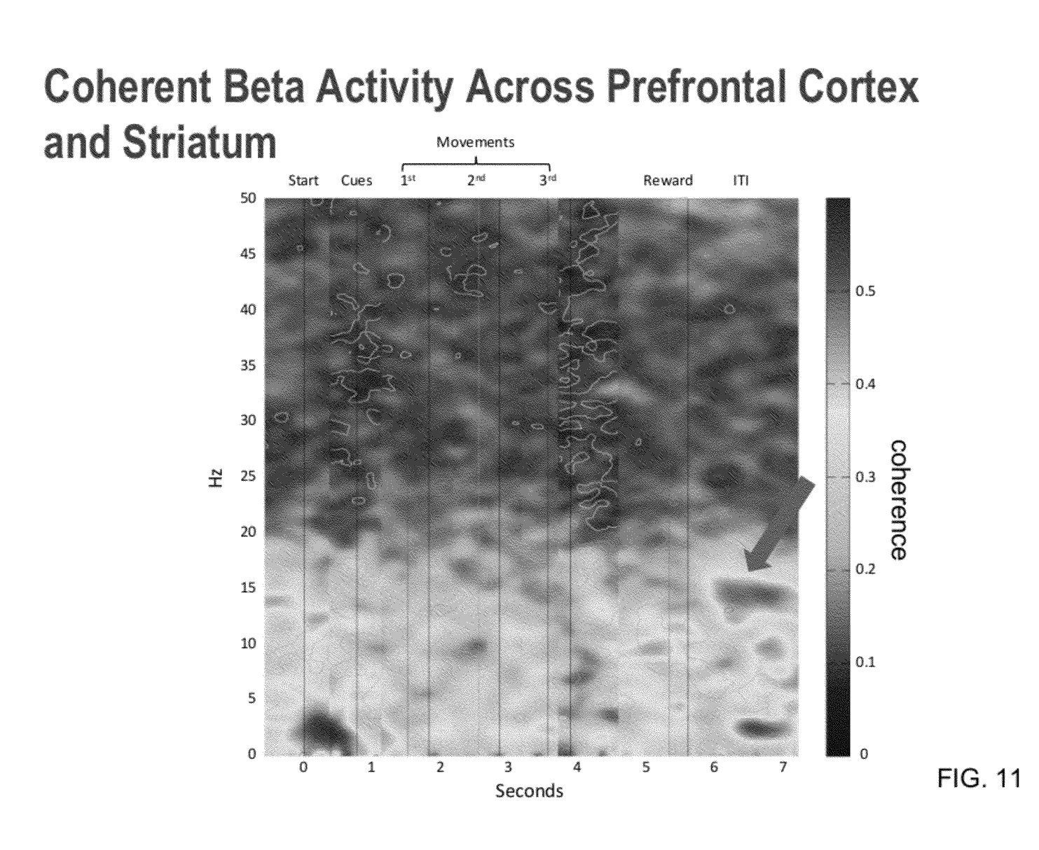

FIG. 11 is an average coherogram across all pairs of simultaneously recorded LFPs in contralateral CN and dlPFC across ˜100 correct trials of the simultaneously cued (Sim) sequential arm movement task. The coherence was computed within a 1 s-long window was stepped 40 ms at a time. FIG. 11 is a composite figure made of 10 event-aligned windows. The peak during ITI around 15 Hz is significantly higher than the coherence at any other point in the trial for this frequency. Low frequency peaks might be significant, but are probably not due to oscillations (small window size). Contour lines represent standard error of the mean (lighter colors correspond to higher values; all are less than 0.05).

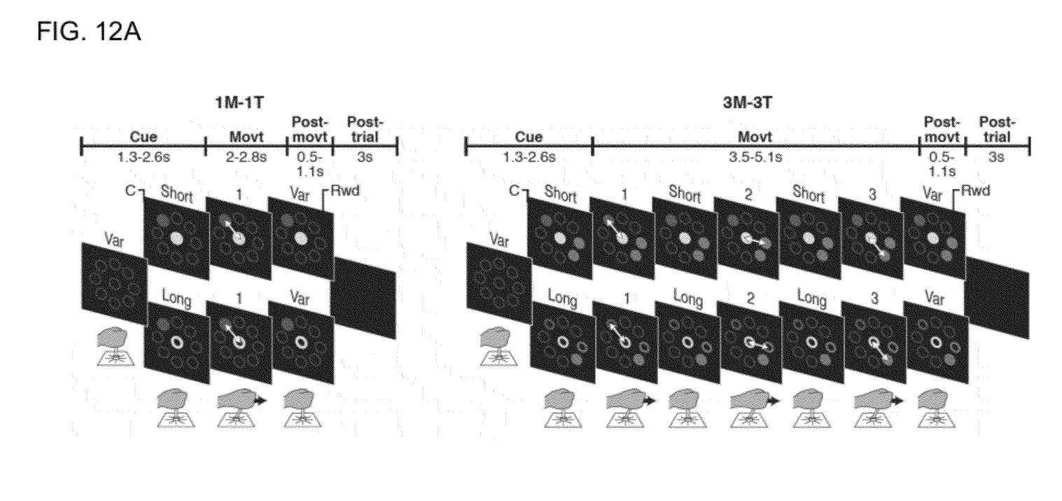

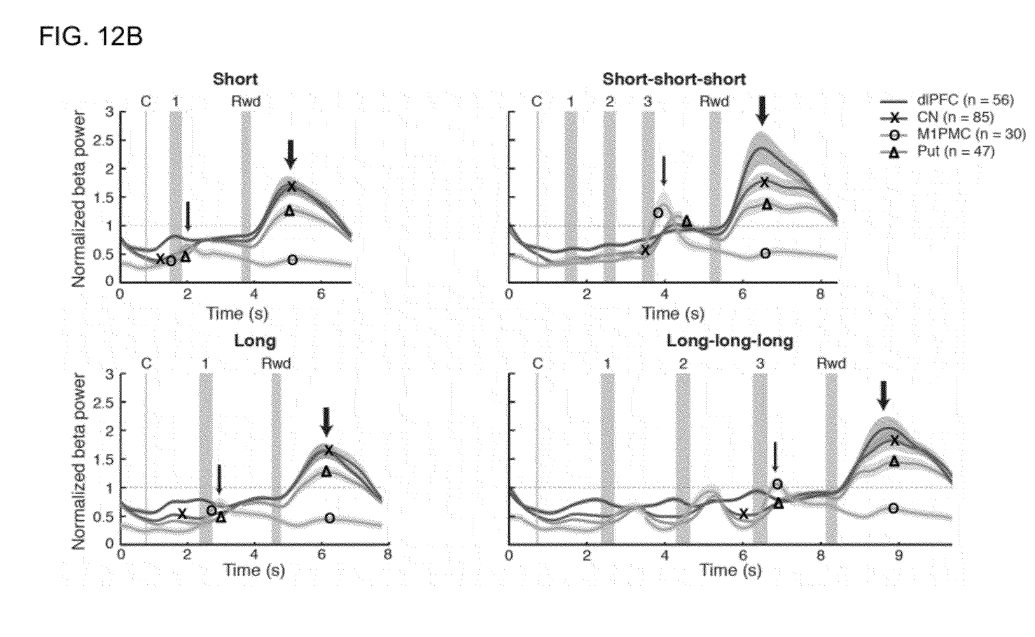

FIGS. 12A-B show prominent post-performance beta oscillations, including a suppression-rebound pattern of the task-modulation of beta power across simultaneously recorded LFPs in M1PMC, dlPFC, CN and Put. FIG. 12A shows a schematic flow of trials of the single (1M1T) or sequential (3M3T) self-timed joystick movement tasks. Timelines show the division of each trial into contiguous periods for analysis (top): cue—from onset of empty cue array until the initiation of joystick movement; movement—from initiation to joystick movement until 700 ms following the offset of the last movement; post-movement—from the end of the movement period until the offset of the visual cues (following reward delivery); and post-trial—from the offset of the visual cues until the start of the next trial (3 s). Each movement was preceded by a short (0.8 s) or long (1.6 s) duration hold period that the monkeys had to self-time. Trials of each task were presented in separate blocks in each experimental session. Following the last movement in a trial, the monkeys held the joystick steady for a variable delay until reward delivery, immediately after which the visual cues disappeared and a 3 s-long post-trial period began. As before, the spatial locations, shapes and colors of the cues, indicated the movement targets, durations of pre-movement hold periods and order of movements, and were changed pseudorandomly from trial-to-trial. Trials of every combination of short and long hold periods were performed in each session.

FIG. 12B shows bandpass-filtered LFP power in the beta band recorded from each electrode was averaged across all correct trials of the short (top left) and long (bottom left) 1M1T tasks, and the short-short-short (top right) and long-long-long (bottom right) 3M3T tasks performed in a single session. The power for each site was then normalized to the average rest value at that site. Results were averaged across the population of LFPs recorded in each brain region in four sessions and plotted in ten windows centered on successive task events (C—onset of the array of visual cues, 1-3—joystick movements, Rwd—reward delivery; pairs of colored traces indicate upper and lower 95% confidence limits).

In addition, LFPs were recorded simultaneously from multiple sites in primary motor and dorsal premotor cortex (M1PMC), dorsolateral prefrontal cortex (dlPFC), caudate nucleus (CN) and putamen (Put) of two Rhesus monkeys. The monkeys had been trained extensively to perform single and sequential joystick movements in response to visual cues (FIG. 12A, 1M1T and 3M3T tasks, respectively).

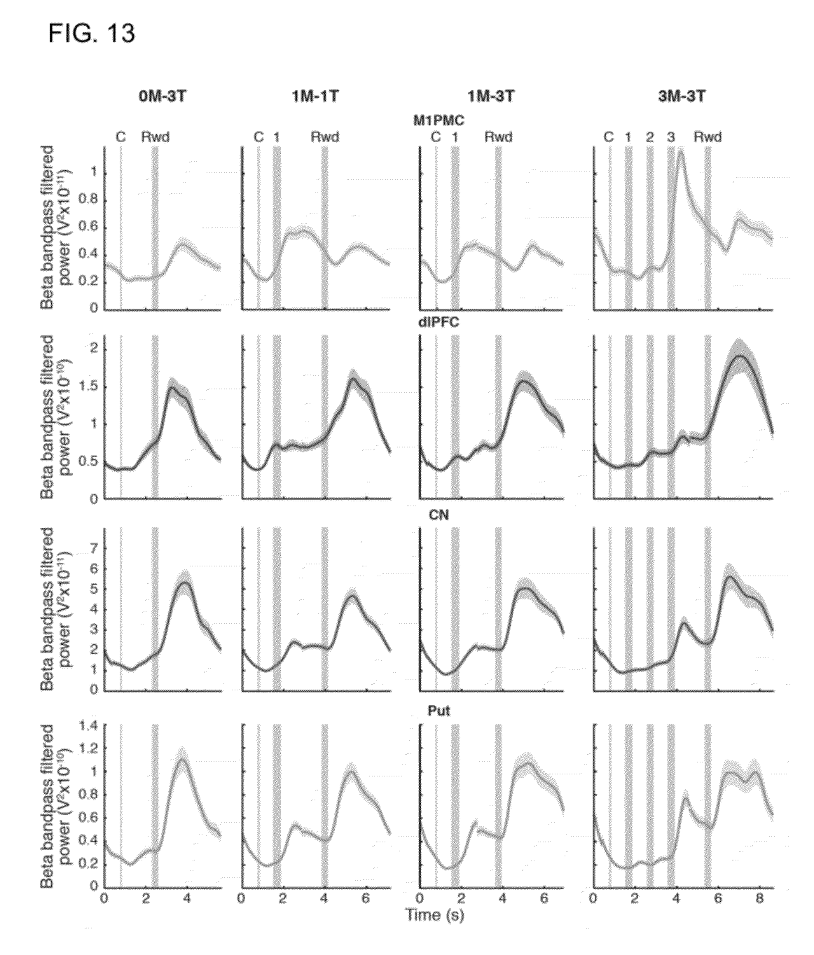

Striking differences were observed between the time-course of LFP power in the beta band across brain regions (FIGS. 12B and 13). During the cue and movement periods, the trial-averaged power in the beta band at each site was suppressed relative to the average value of the beta power at that site recorded during prolonged rest periods (FIG. 12B, dashed lines) before and after behavioral task performance. As expected, in M1PMC, the trial-averaged beta power during the 1M1T task reached a maximum immediately following the offset of the movement (FIG. 12B, thin arrows). Interestingly, the highest peak in beta power during the 3M3T task occurred following the offset of the last movement in the sequence, not following each movement. Additional minor peaks were observed following the first and second movements, but only during long hold periods. Thus, the peak in beta power in M1PMC was not locked to the offset of any given movement per se, but rather to the offset of the last movement in a sequence. This suggests that beta activity in M1PMC may be related to the completion of, and subsequent disengagement from, the performance of a motor task.

The timing of the rebound in power in dlPFC and striatum was dramatically different from what it was in M1PMC. Instead of peaking in the post-movement period, as in M1PMC, beta power in dlPFC and striatum peaked in the post-trial period, following reward delivery and the subsequent offset of the visual cues (FIG. 12B, thick arrows). This difference in the timing demonstrates that beta activity is spatially localized, at least between different brain regions, and further shows that beta activity is not simply locked to movement onset or offset.

These results argue against a simple relationship between movement and the modulation of beta activity, even in M1PMC. Nevertheless, they were obtained during the performance of tasks requiring overt motor responses. Does the suppression-rebound pattern of modulation in beta power occur only during trials involving movement? To answer this question, the timing of the peaks in beta power was analyzed during trials of a third task, 0M3T, in which the monkeys were presented with the same visual cues as in the 3M3T task (FIG. 12A), but were required to withhold movement in order to obtain reward. Remarkably, in the 0M3T task, the beta power in each of the four brain regions exhibited a pattern of peri-cue suppression, followed by a rebound during the post-trial period (FIG. 13).

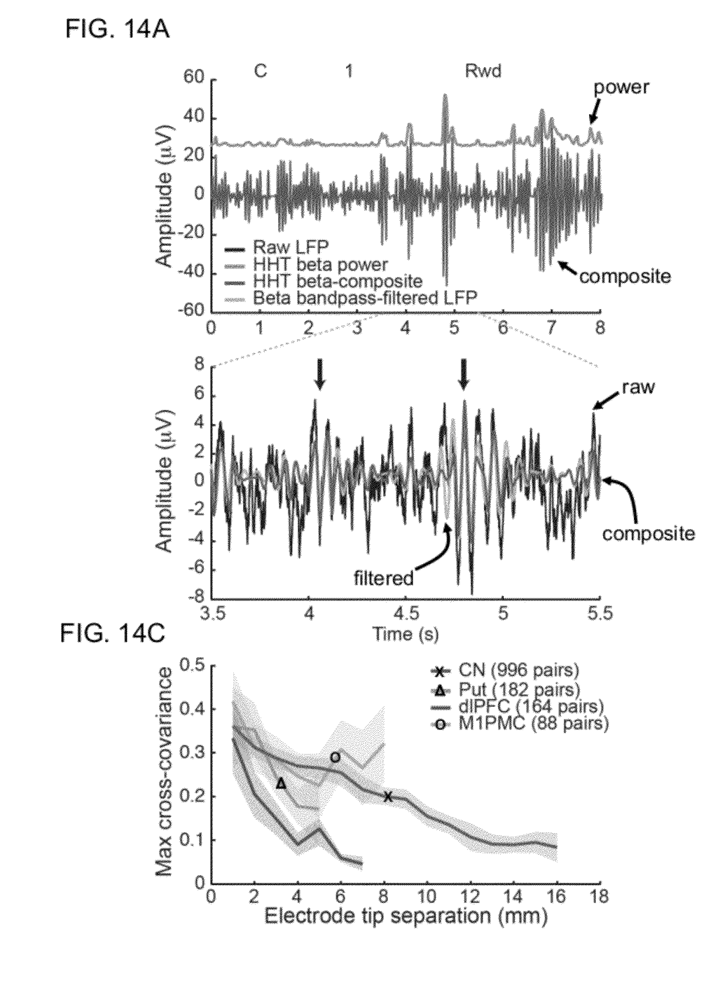

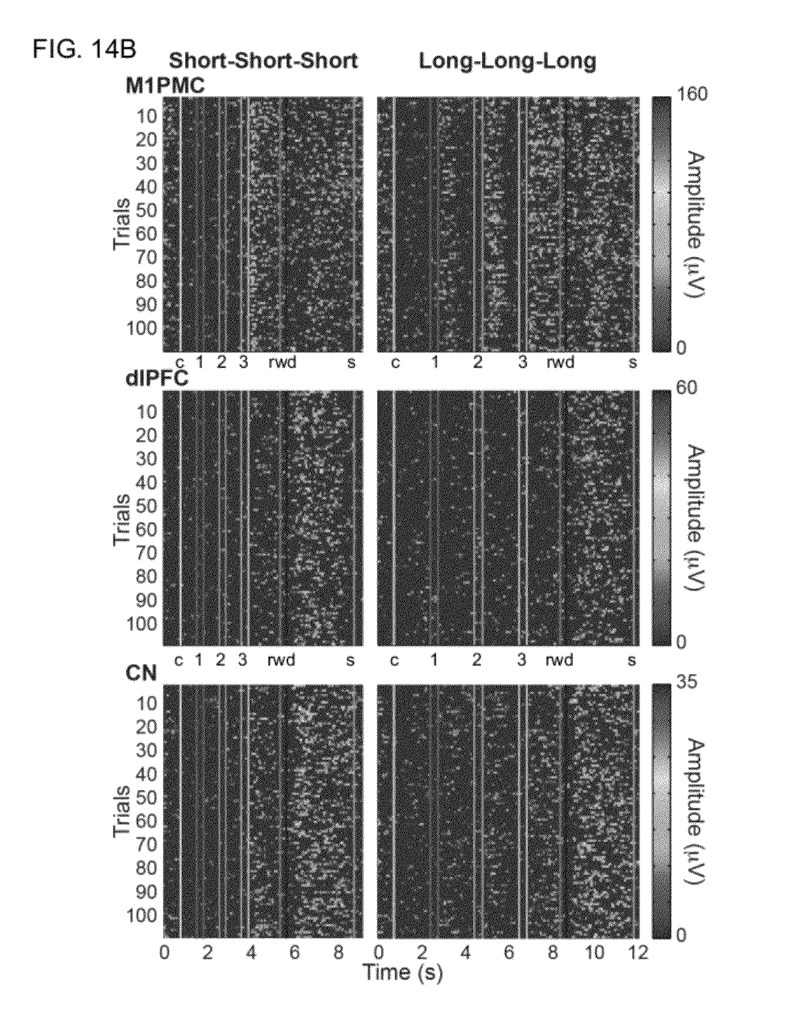

FIGS. 14A-C show spatially localized bursts of beta oscillations in M1PMC, dlPFC, CN and Put. FIG. 14A shows the beta band content of the Hilbert-Huang transform (HHT) of an LFP recorded in a single 1M1T trial (top) exhibited short episodes during which it accounted for a relatively large percentage of the spectrum of the raw LFP (bottom, black arrows). The beta bandpass-filtered waveform (bottom) showed similar modulation to the HHT, but with considerably less accuracy (in terms of faithful representation of beta oscillations in the raw LFP). FIG. 14B shows beta bursts varied in amplitude and timing across correct trials of the short-short-short and long-long-long 3M3T task in three simultaneously recorded LFPs in M1PMC, dlPFC and CN. The rate of beta bursts at each site varied systematically in relation to task events, following a similar time-course to that of the trial-averaged beta power at the same site. Trials began at time 0. Vertical lines indicate task events (from left to right, c—onset of visual cues, 1, 2 and 3—1^{st}-3^{rd} joystick movements, rwd—reward delivery, s—start of next trial). FIG. 14C shows the maximum cross-covariance between the envelopes of beta bursts recorded at pairs of sites was averaged across all pairs within each brain region (thick lines—means; shading—95% confidence limits). For inter-electrode distances of <1.5 mm the values of the cross-covariance were not significantly different across brain regions. However, at greater distances, the average values for dlPFC pairs fell more quickly than the values for the other brain regions.

The time-course of the trial-averaged beta power in relation to task performance suggests the existence of sustained beta oscillations either post-movement (in M1PMC) or post-trial (in dlPFC and striatum). Sustained oscillations, if present during periods of rest or “steady-state” behavior, would support current interpretations of beta activity as an indicator of idling or of state-preserving processes. However, the results so far have been based on trial averages. In order to detect the occurrence of sustained beta oscillations during task performance, individual trials were analyzed. Surprisingly, no single trial resembled the trial average, in that there were no periods of sustained high-amplitude beta oscillations. Rather, each trial was characterized by brief (˜150 ms) episodes during which beta frequency oscillations dominated the spectrum (FIG. 14A). These bursts of oscillations in the beta band were detected based on their relative contribution to the spectrum of the LFP signal, as opposed to their overall amplitude. In each brain region, beta bursts occurred throughout the trial, even during movements. However, the modulation of the burst rate in each region closely followed the modulation of the trial-averaged beta power in that region (FIG. 14B). This led us to reinterpret the trial-averaged beta power as expressing the time-dependent probability in any given trial that a beta burst will occur. The large trial-to-trial variability in burst amplitude and duration, as well as in the timing of the bursts relative to task events, gives rise to the temporally extended peaks in the trial-averaged power.

The bursts in the beta composite HHT were detected based on the relative contribution of the composite (at each time point) to the broadband LFP signal (5-50 or 5-100 Hz).

When the contribution rose above a given threshold, potential bursts were marked. Burst boundaries were detected by proceeding forward and backward in time from each supra-threshold local maximum in the contribution, until any of three constraints were violated: the phase of the composite HHT shifted at a rate above an allowable threshold; the composite HHT failed to cross zero between successive extrema; or the level of the contribution of the composite to the broadband LFP fell below an allowable threshold.

All thresholds were computed on a per electrode basis, electrode by electrode, based on comparing the recorded LFP on each electrode to the composite HHT of simulated pink noise.

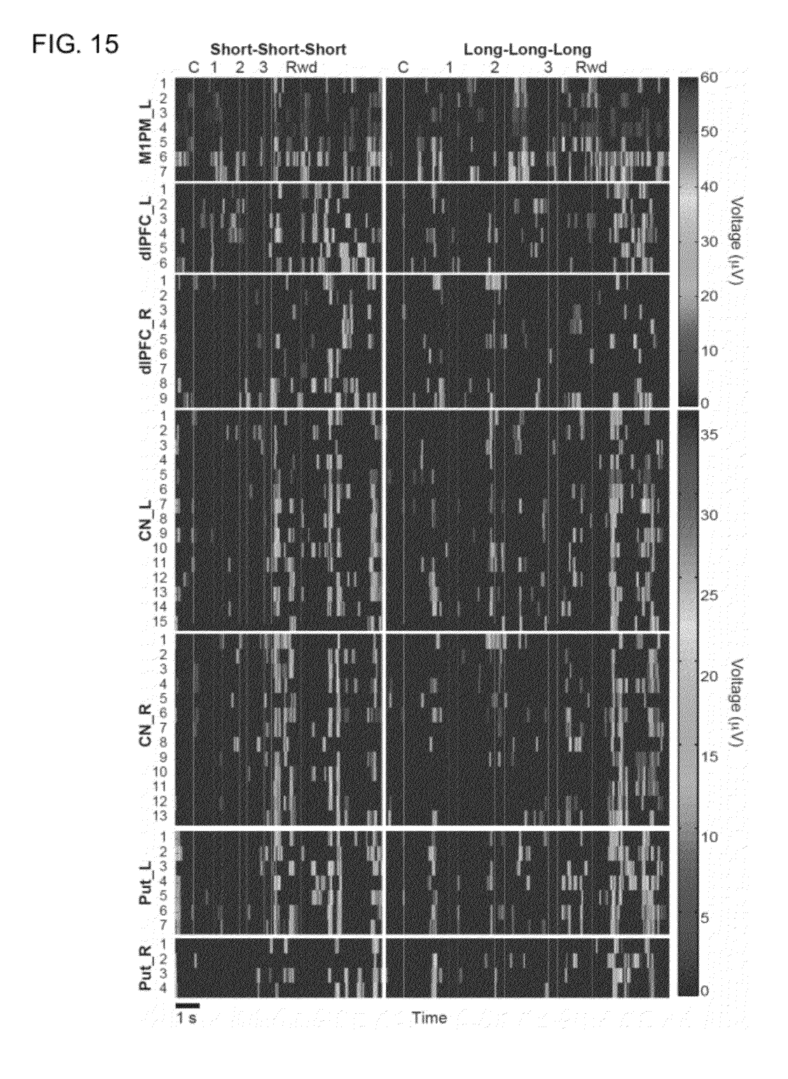

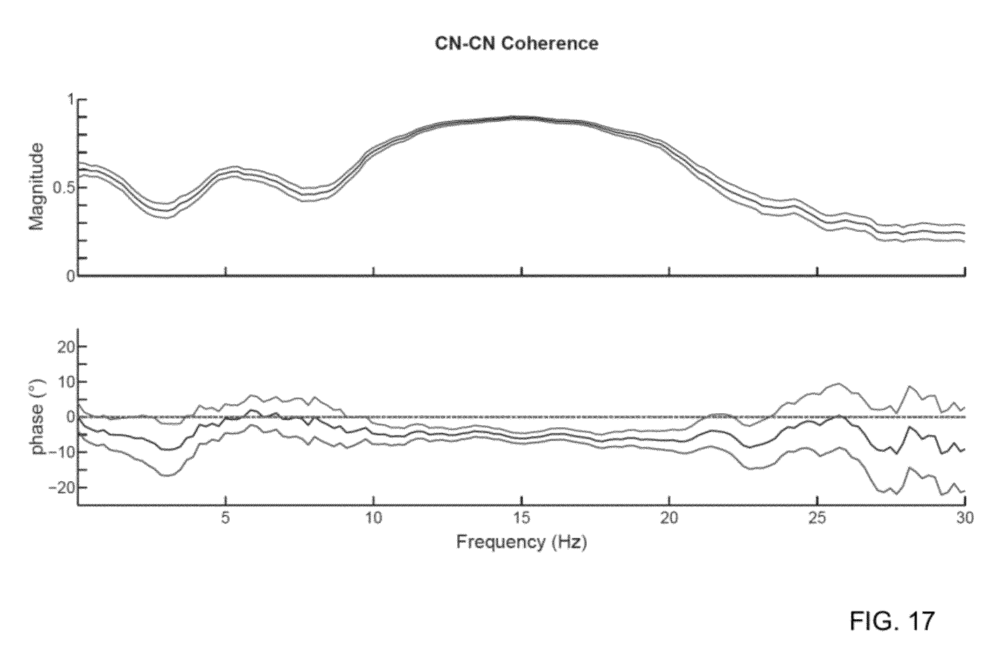

Beta oscillations were not only temporally discrete, but were also localized in space. In each of the four brain regions we studied, the peak cross-covariance between the envelopes of beta bursts recorded at pairs of sites decreased significantly as the distance between the recording sites increased (FIG. 14C; unless otherwise noted, statistical significance was assessed using an analysis of variance (ANOVA) statistical test corrected for multiple comparisons, (alpha(significance level of 95%)=0.05). The degree to which bursts were spatially localized differed across brain regions, with dlPFC exhibiting significantly greater localization of bursts than M1PMC and the striatum for distances of 1.5 mm or more between paired sites. The differences between regions in the spatial localization of bursts can be seen in individual trials, by comparing the bursts across the population of simultaneously recorded sites across the four brain regions (FIG. 15). Importantly, the phase of the coherence between bursts in the LFPs from even the closest pair of simultaneously recorded electrodes in the CN was significantly different from zero (FIG. 17), arguing against the possibility that electronic volume conduction between the sites could have accounted for the high cross-covariance observed between bursts. These results demonstrate that cortical and striatal beta activity occurs in the form of spatiotemporally discrete episodes, the modulation of which varies from trial-to-trial and from site-to-site.

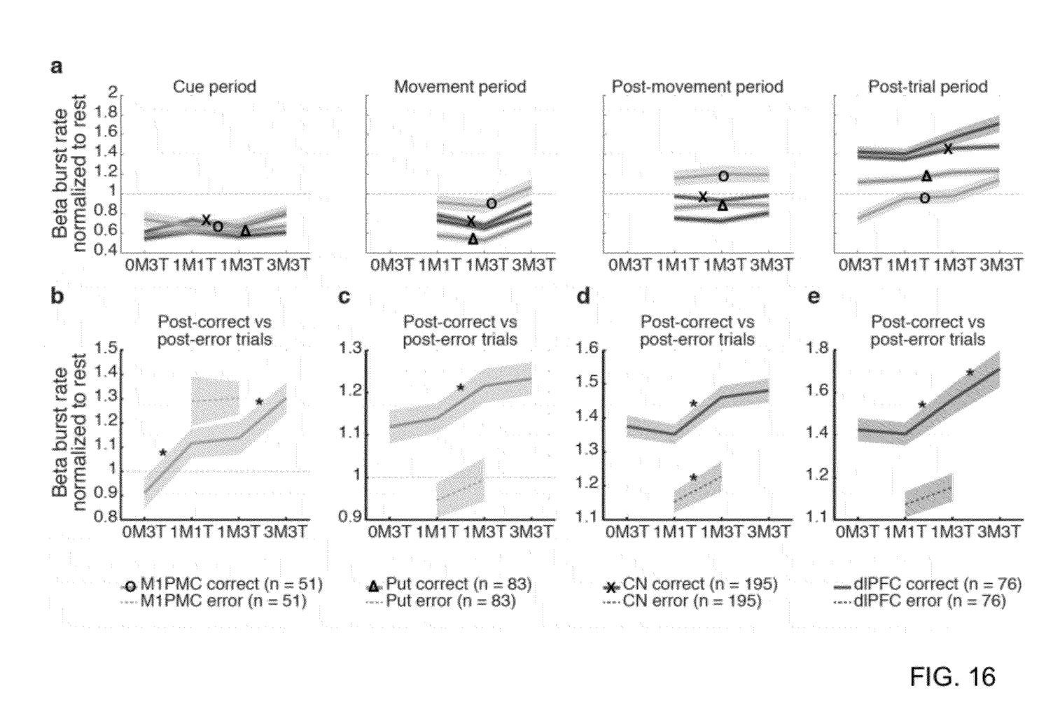

FIGS. 16A-E show differential modulation of population beta burst rates in M1PMC, dlPFC, CN and Put by behavioral tasks. FIG. 16A shows the beta burst rate for each LFP in each task and task period was averaged across trials. The resulting burst rates for each LFP were normalized by the average value during rest periods for that LFP, and then the burst rates were averaged across the population of LFPs recorded in each brain region, across all sessions (horizontal dashed line denotes population average burst rate during rest periods). The population-average of normalized burst rates in each brain region followed a pattern of modulation that was similar to the time-course of beta band power in that region. There were no data points for the movement and post-movement periods in the 0M3T task. FIGS. 16B-E show population average normalized burst rates shown in each brain region across tasks during post-trial periods following correct and error trials (thick solid and thin dashed lines, respectively; shading—95% confidence limits). The values for correct trials were re-plotted from right-most plot in FIG. 16A with different vertical scales. Asterisks indicate statistically significant differences between adjacent pairs of data points in correct trials or separately in error trials (ANOVA corrected for multiple comparisons, alpha=0.05). Statistically significant differences between correct and error trials are demonstrated by non-overlapping shading (95% confidence intervals) between the thick and thin lines.

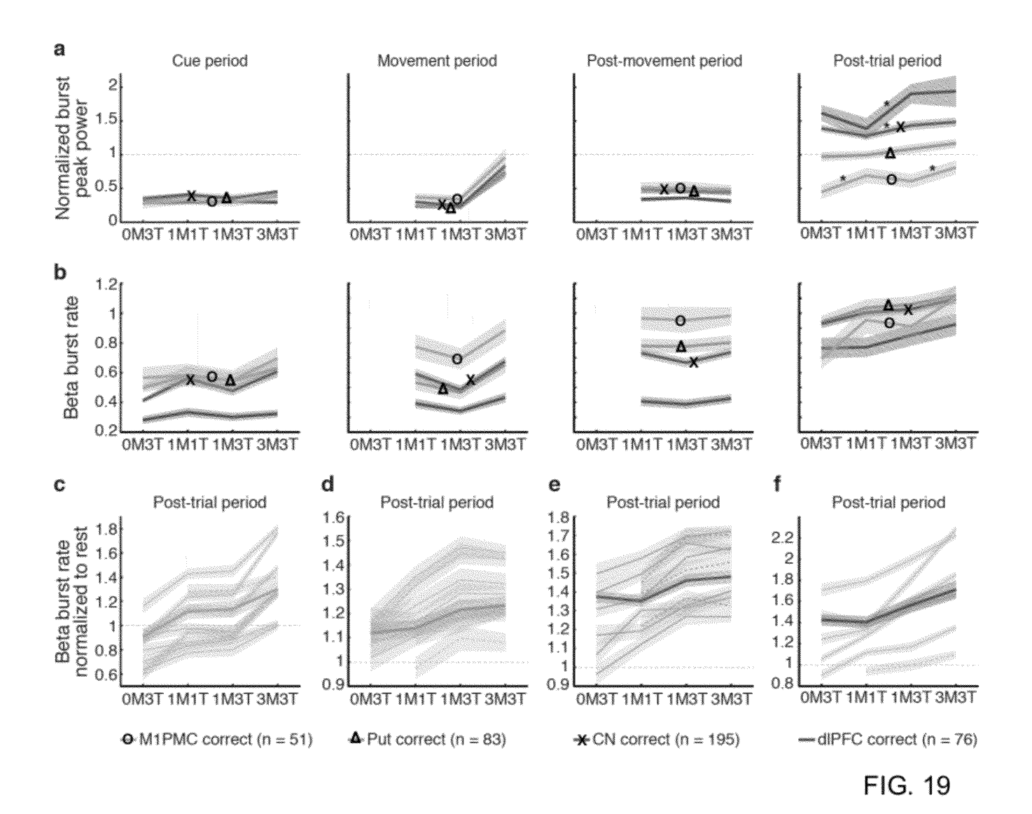

No evidence was found for waxing and waning beta oscillations, but rather, a time-dependent probability of beta burst occurrence that varied by brain region (FIG. 16A, 1M1T and 3M3T tasks). For each site, the rate of bursts were calculated in each task period relative to the average rate during rest periods at that site, and the rates were averaged across all sites within each brain region. The modulation of the population average burst rates in each brain region followed the time-course of the trial-averaged beta band power in all behavioral tasks (FIG. 16A; compare to FIG. 12B). During the cue and movement periods, beta burst rates were suppressed relative to the average value during rest periods. The rest-normalized burst rates then peaked in M1PMC post-movement, whereas in dlPFC and striatum, the burst rates peaked post-trial. The population average burst power (normalized to rest) followed a similar pattern of modulation across task epochs (FIG. 19A). In addition to the burst rates that were normalized to rest, we examined the modulation of the absolute burst rates (FIG. 19B). Although the highest normalized bursts rates in the post-trial period were in the dlPFC, this region showed the lowest absolute burst rates. The converse was true of the M1PMC bursts. Thus, across brain regions, there was an inverse relationship between the rate of bursts during rest and the degree to which those rates changed following trial performance.

We tested whether beta burst rates and power during the post-trial period were modulated by specific aspects of the preceding trial. For this purpose, a fourth behavioral task, 1M3T, was added in which the monkeys were presented with the same visual cues as in the 3M3T task, but were required to perform only the first instructed movement in order to obtain reward. The rate and, separately, the power, of beta bursts were compared following trials of four behavioral tasks (performed in separate blocks of each experimental session), involving different numbers of movements (0M3T, 1M3T and 3M3T) or of visual cues indicating the potential spatial targets of movement (1M1T and 1M3T). In each brain region, the population average of normalized rates of beta bursts in the post-trial period were modulated significantly by the details of the preceding behavioral task performance (FIGS. 16B-E, asterisks indicate significant differences between burst rates in adjacent tasks). The population average of normalized beta burst power in the post-trial period matched the pattern of burst rate modulation across tasks (FIG. 19A, right-most panel).

Not only were the post-trial burst rates modulated by the preceding trial type, but the pattern of modulation differed across brain regions. In M1PMC, burst rates showed significant modulation between tasks with 0, 1 or 3 movements, regardless of the number of visual targets (FIG. 16B). Thus, the rate of bursts in M1PMC during the post-trial period tracked the number of movements that the monkey had performed in the preceding trial. This is surprising given that beta activity (both rate and power) in M1PMC during this time period was below the peak levels attained during the immediate post-movement period, and was, in fact, close to rest levels. In contrast to these burst patterns in M1PMC, the pattern of beta burst rate modulation in CN and Put during the post-trial period across different tasks indicated a significant effect of the number of visual cues, as opposed to the number of movements, in the preceding trial (FIGS. 16C,D). This relationship between striatal burst rates and the number of visual cues was consistent so long as the monkey was performing a movement task, as opposed to withholding movement (in the 0M3T task). Finally, the burst rates in dlPFC increased with increasing numbers of movements and of visual cues instructing movement (FIG. 16E). Importantly, individual LFPs in each brain region exhibited patterns of task-dependent modulation of post-trial burst rates that were qualitatively similar to the patterns found for the population averages (FIGS. 19C-F, thin lines). This agreement between the results for individual LFPs and those for the population average confirms that post-trial bursts in localized sites in each brain region were modulated by the same aspects of the preceding behavioral task performance that modulated the population average burst rates.

To determine whether the post-trial burst rates depended on the outcome of the trial, we tested whether beta burst rates following correct trials differed from those following error trials (FIGS. 16B-E, solid thick vs. dashed thin lines, respectively). In order to control for possible differences in the number of movements between correct and error trials, single-movement tasks were focused on, and were analyzed for only those error trials in which the monkey had performed a single movement (the monkeys could have made a movement that resulted in an error trial either by initiating the movement too quickly or by making the movement in an incorrect direction; min. of 20 error trials per condition). In dlPFC and striatum, the burst rates following error trials were significantly lower than those following correct trials (FIGS. 16C-E, solid vs. dashed lines). In contrast to the other brain regions, the burst rates in M1PMC following error trials were significantly higher than those following correct trials. In fact, the burst rates in M1PMC following error trials were indistinguishable from those in the post-movement period in correct trials. This is not surprising, given that, in terms of the timing relative to the offset of movement, the post-trial period following error trials coincided with what would have been the post-movement period in correct trials. This fact indicates that, unlike striatum and dlPFC, the M1PMC burst rates in the post-trial period were not modulated by the overall outcome of task performance.

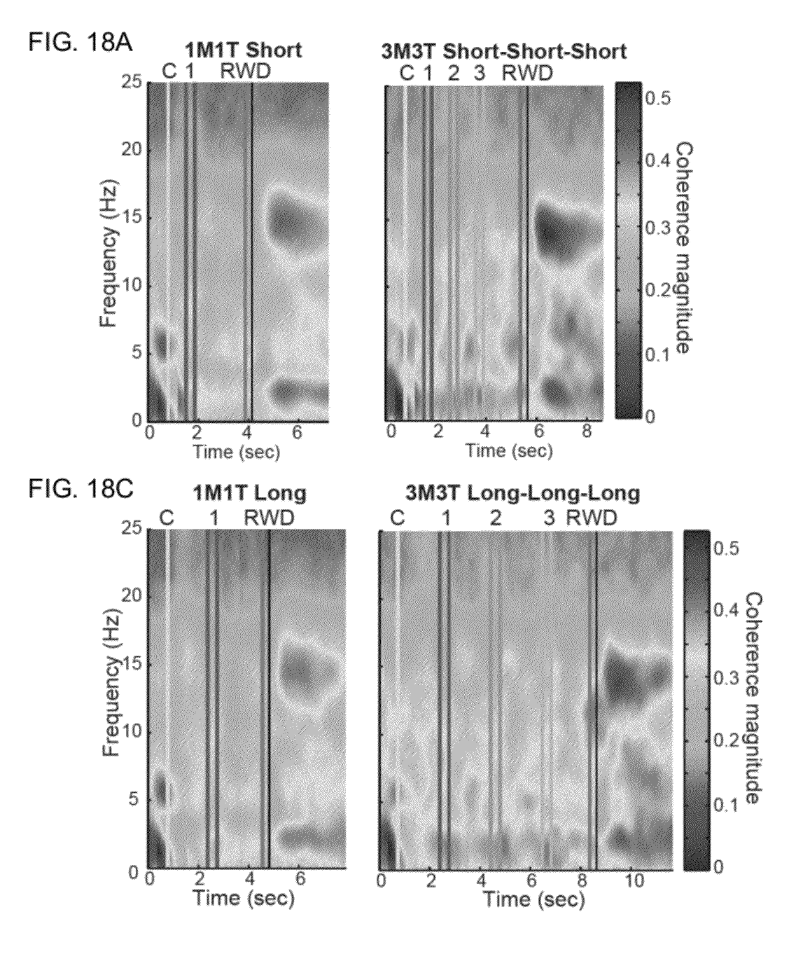

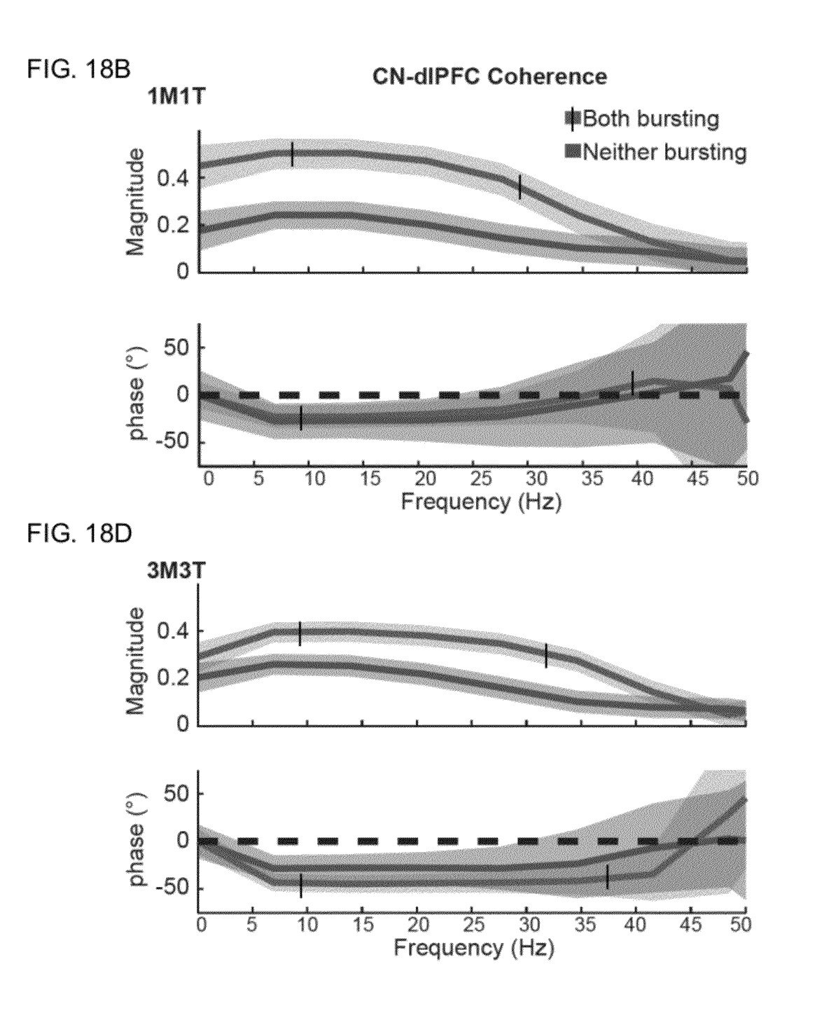

FIGS. 18A-D shows beta range coherence between dlPFC and CN is highest in the post-trial period and disproportionately due to bursts. FIG. 18A shows population average coherograms across all pairs of simultaneously recorded LFPs in dlPFC and CN across all sessions. Coherence magnitude is shown in pseudocolor across correct trials of the short 1M1T and short-short-short 3M3T tasks. The values, shown in windows aligned on the task events, indicate a significant peak in the beta band (˜15 Hz) during the post-trial period. FIG. 18B shows an example from a single pair of CN-dlPFC LFPs, showing increased coherence in the post-trial period when both LFPs are bursting, as opposed to when neither is bursting (top plot; thick lines—means, shading—95% confidence limits). The phase of the beta band coherence in both cases was significantly lower than zero (bottom plot). The striatal LFP led the prefrontal one by ˜8 ms. FIG. 18C shows the same graph as FIG. 18A but for long 1M1T and long-long-long 3M3T tasks. FIG. 18D shows the same graph as FIG. 18B for all post-trial periods following correct trials of the 3M3T task.

Given that, in all tasks, the rate and power of beta bursts in the dlPFC and striatum were highest during the post-trial period, we asked whether the during this period the bursts at pairs of striatal-prefrontal sites exhibited consistent temporal relationships, which they did. The population average coherence between all pairs of simultaneously recorded LFPs in the CN and dlPFC reached a peak in the beta frequency range during the post-trial period, following trials of the 1M1T and 3M3T tasks (FIGS. 18A,C). The coherence values at these post-trial peaks were significantly higher than the coherence in any other trial period. Given the prominent bursting at beta frequencies during the post-trial period, we asked whether the high coherence during this period might be due to elevated coherence specifically between beta bursts. Indeed, individual pairs of recording sites in the CN and dlPFC showed significantly higher post-trial coherence during periods in which both LFPs were bursting, than when neither was bursting (FIGS. 18B,D). The significantly non-zero phases of the coherence in either case rules out the possibility of volume conduction between the two sites or from a common third site. At the population level, the ratio of the magnitudes of coherence among pairs of LFPs that were bursting as opposed to not bursting was ˜3, and was significantly higher during the post-trial period in all tasks than in rest periods. This ratio differed significantly across tasks, and was inversely related to the number of movements performed in the preceding trial.

In addition to the temporal relationships between bursts in different brain regions, co-activation of bursts were studied across different sites within each region. These results are preliminary, based on data from a single experimental session. Our analysis took into account the entire population of simultaneously recorded LFPs within each region, as opposed to averaging results across all pairs of LFPs across regions. Beta bursts in the striatum were significantly more co-active than beta bursts in M1PMC and dlPFC. This fits well with the earlier results of the cross-covariance between bursts at pairs of increasingly distant sites (FIG. 14C), as well as examples of bursts across the population of simultaneously recorded sites in individual trials (FIG. 15). In M1PMC and striatum, the co-activation of bursts in the post-trial period (normalized to the co-activation during rest periods) was modulated across tasks in ways that were similar to the task-modulation of the population average burst rates. Remarkably, the post-trial bursts in dlPFC, in contrast, exhibited a pattern of modulation across tasks that was opposite to the corresponding pattern of burst rate modulation.

LFP-LFP coherence between CN and dlPFC showed a significant peak during periods in the ITI. Coherence among coincident signals is a strong indicator of potential communication between sites. Therefore, each pair of simultaneously recorded LFPs were analyzed across the two brain regions (one in CN and the other in dlPFC), when either both electrodes of the pair were bursting or both were non-bursting. The majority of electrode pairs showed significantly greater coherence in the beta band when they were bursting than when they were non-bursting, suggesting that the bursts themselves were coherent with each other, as opposed to a general coherence between the LFP signals. This further supports the interpretation that beta bursts are related to post-task performance communication or concerted updating across widely distributed networks in the brain. This result, coupled with our earlier finding of increased bursting following correct, rewarded task performance, as opposed to erroneous, unrewarded performance, suggests that beta bursts represent concerted network activity following “successful” behavior.

Based on these results, the invention provides methods for tracking the changes in the beta frequency range of EEG, MEG, ECoG or LFP activity (or similar brain activity signals), recorded from any part of the human or animal brain, in order to detect the engagement and disengagement of individual brain regions during the performance of motor or non-motor tasks—that is, even tasks that are purely cognitive, without any overt motor output. In some embodiments, the method involves the following steps:
1. Analyze brain activity signals (EEG, MEG, ECoG or LFP) in order to detect bursts in the beta frequency range (13-30 Hz), using the HHT along with algorithms for constructing a measure of the beta-band component of the signal, which we call the beta composite, from the Intrinsic Mode Functions of the HHT, and for detecting bursts in the beta composite based on the goodness-of-fit of the beta composite to broadband brain activity and other criteria (sinusoidal-like behavior, and phase and frequency constraints). Beta bursts can be detected in near-real-time (<0.5 s lag), using a digital implementation of our modified HHT, or in real-time using conventional filtering techniques to monitor continuously the proportion of total signal power that falls within the beta band.
2. Analyze the parameters of the beta bursts at individual brain sites (including timing, amplitude, duration, frequency, rate, phase, envelope and power), in order to determine the boundaries and depth of task engagement for each brain site.
3. Analyze the concurrence and coherence between beta bursts at simultaneously recorded brain sites, in order to assess the synchronization of bursts, as an indicator of potential communication and information transfer between brain sites during and following task engagement.

The detection of beta bursts in brain activity signals can be used to measure quantitatively the brain-site-specific depth of engagement, e.g., based on post-engagement burst rates and amplitudes, as well as the duration of engagement—the neural processing time each brain region devotes to the task—based on the timing of significant changes in burst parameters (with low and high burst rates corresponding to task engagement and disengagement, respectively). Both the depth and duration of brain-site-specific task engagement can be used as important physiological measures of performance and learning, complementing current behavioral measures such as reaction time and the percentage/frequency of errors. Clearly, this monitoring system can be used as a training tool, and can aid in focusing therapeutic interventions to the times that are relevant for such interventions, in conditions ranging from ADD, ADHD and autism spectrum disorders (ASDs) to conditions involving overt problems with movement or thought preparation and cessation, including OCD, PTSD and learning disabilities.

This detection system can be used to analyze individual differences in performance and learning. Individuals might break up complex problems into a series of smaller component tasks, which can be detected on the basis of beta power modulation as we have found in our non-human primate experiments. This detection would provide an independent measure of engagement and task-decomposition that can be useful in addition to reaction time measures, known to be inadequate markers in many circumstances. Detection of the beta burst episodes and their patterns can also be useful for detecting when a subject might be “zoning out” while performing a task of extended duration, and thus can have high relevance for contexts ranging from air traffic control to vehicle operators to student groups.

Beta bursts, though spatially localized, can be significantly coherent across particular brain regions, a finding highly suggestive of inter-area communication. Thus, the detection system can be used to detect abnormal communication during behavior, e.g., in autism or schizophrenia.

We have shown significant differences across brain regions in the timing of peak beta activity relative to behavioral events. The timing of the peak in each brain region was consistent with a role for beta oscillations in post-performance processing of behavior. Investigating the source of the peaks in the trial-averaged beta power, we discovered that beta activity was fundamentally characterized by brief, spatially localized bursts of oscillations, whose rate and power were modulated by the behavioral tasks in a manner similar to the modulation of trial-averaged beta power. The modulation of post-trial bursts by task features (numbers of movements or visual cues) differed across brain regions, as follows: M1PMC bursts tracked numbers of movements, striatal bursts tracked numbers of cues and prefrontal bursts tracked a combination of both. Post-trial beta burst rates in striatum and dlPFC were also modulated by the outcome of the preceding behavioral task performance—burst rates were significantly higher following rewarded correct trials, as opposed to unrewarded errors. During the post-trial period, beta band coherence between pairs of brain regions reached a maximum. Paired sites in CN and dlPFC exhibited increased coherence specifically during beta bursts, consistent with the view that the bursts may facilitate communication or coordination across brain regions. This idea is supported by computational work showing that beta oscillations are particularly well-suited to long-range interactions, as well as by recent physiological work on task-dependent changes in beta coherence across cortical regions.

Taken together, our results suggest a unifying interpretation of beta oscillations in the brain. Our findings argue against the view that beta activity prevents brain regions from changing their present activity, and hence, the current behavioral output. Rather, we propose that beta oscillations represent mechanisms for integrating the successful outcome of behavior with the details of the performance that led to that outcome, across cortico-striatal networks. The purpose of such mechanisms could be to increase the likelihood of achieving goals in future trials, in either of two ways: either by preventing changes to network activity that led to a rewarded outcome, or by actively reinforcing such network activity.

Such a role for beta oscillations is consistent with current ideas about their mechanistic origins. In frontal cortex and basal ganglia beta oscillations are thought to arise from the interplay of excitatory and inhibitory feedback. In the basal ganglia, computational work has identified the GPe-STN network as a likely candidate generator of beta oscillations, which can arise from the inhibition of STN by GPe, coupled with the excitation of GPe by the STN. In vitro slice work suggests that beta oscillations in sensorimotor cortex may reflect gap-junction-dependent firing of pyramidal cell layer neurons. Furthermore, recent computational work has demonstrated that beta oscillations in association cortex can sustain representations of stimuli in short-term memory for the purpose of integrating changing stimuli across time. It is possible that beta activity in other brains regions could similarly function to integrate behavioral outcomes with the details of the behavior that led to them, and then tune or update network activity accordingly.

Low beta activity following an unrewarded outcome may allow synaptic plasticity to occur, mediated by other mechanisms. The purpose of such plasticity could be to change the network dynamics in order to increase the likelihood that the desired outcome will be obtained in the future. This would be particularly useful in learning situations (exploration), and warrants further study of the potential evolution of beta activity during learning. On the other hand, in the performance of well-learned behaviors, low beta activity following occasional errors, as we have observed in our experiments, might prevent the inappropriate reinforcement of the network activity that led to the undesired outcome. In such a scenario, low beta would effectively prevent the occasional erroneous performance from modifying the monkey's typically successful behavior. Beta activity would thus maintain the learned network settings by making them less susceptible to change by infrequent errors (exploitation). Thus, rather than promoting the current behavioral output, as suggested by the status quo-preserving theory, beta oscillations could be involved in promoting the current network settings that generated the behavior that culminated in a successful outcome. This would serves to increase the likelihood that the same outcome would be obtained in similar situations in the future.

Patients with PD, a disease involving a loss of the major source of dopaminergic input to the striatum, exhibit pathologically high coherence in beta band activity in the cortex and basal ganglia. While the clinical consequences of elevated beta synchrony are not clear, evidence has been mounting that it can be reduced by the leading treatments for PD. Remarkably, recent studies have shown that decreases in beta activity in the subthalamic nucleus can correlate with decreases in motor symptoms of PD, specifically bradykinesia (slowness of movement) and rigidity. Other work recently established a link between high cortical beta power and bradykinesia, and some have proposed that the benefits of deep-brain stimulation therapy are mediated by reducing high beta synchrony. Patients with PD typically present with cognitive as well as motor symptoms. Our results indicate a possible causal relation between the abnormally high beta activity in the cortex and basal ganglia of PD patients and a specific type of learning deficit associated with PD. A recent study found that PD patients were more likely than normal subjects to perseverate in their choices, independently of reward history, and that this perseveration in choice decreased with dopaminergic therapy. Notably, beta activity in PD patients has also been shown to decrease with dopaminergic treatment. The indiscriminately high beta activity in PD patients could drive their choices by reinforcing the behavior that led to the previous choice, regardless of whether it was rewarded. Alternatively, the high beta activity might prevent the plasticity necessary for the brain to learn from an erroneous choice, modify the network settings accordingly, and increase the likelihood of choosing correctly in future trials. We predict that the direct manipulation of beta activity in PD patients would reduce both akinesia and the perseveration of choice errors.

Particularly interesting are the unique features of beta activity in the dlPFC. Despite the fact that across all tasks the dlPFC had the lowest rate of bursts among the four studied brain regions, during the post-trial period it exhibited the greatest change in burst rates relative to rest (FIGS. 12B and 16A). Furthermore, the dlPFC alone exhibited modulation of post-trial burst rates by both the number of visual cues and by the number of movements in the preceding trial. The greater the number of visual stimuli, and the greater the number of the responses the monkey made to them, the higher the post-performance burst rates were in dlPFC. Thus it appears that dlPFC bursts in the post-trial period were tracking the cognitive load during the preceding trial. Furthermore, the preliminary results of our population-wide burst co-activation analysis indicate that not only were prefrontal burst rates in the post-trial period more spatially localized than they were in the other brain regions, but these bursts became increasingly more localized with increasing cognitive load during the preceding trial, even though the burst rates increased. This stood in contrast to the results from the other three brain regions, in which post-trial burst rates and the level of within-structure co-activation went hand-in-hand. Taken together, these results are consistent with the accepted role of dlPFC in executive control, and support our view of the involvement of beta activity in the post-performance coordination of neural activity across multiple sites in the brain.

A measure of burst concurrence was developed to quantify the degree to which beta bursts tend to occur simultaneously on multiple electrodes, which is computed as follows. Individual shuffled pseudo-trials were constructed by choosing trials at random without replacement across the set of N electrodes being analyzed, so that no two channels contained data from the same trial. A large number of such shuffled pseudo-trials was constructed and used as a proxy data set with the same statistics as the original data set, but with any correlations across channels destroyed. Then, a histogram of the frequency was computed with which each possible number of bursts from 0 to N occurred simultaneously at each time point in the proxy data set, and the thresholds were found for the high and low tails of the histogram that included no more than 2.5% of the pseudo-trials in each tail. Since the thresholds were constrained to be integers, we computed the actual fraction of pseudo-trials contained in the bins beyond the thresholds. The same type of histogram was constructed for the original data, and “concurrence” was defined as the ratio of the fraction of beyond-threshold trials in the original data set to that in the randomized proxy data set. A concurrence value of 1 thus corresponds to chance (i.e. uncorrelated) simultaneous bursting and/or simultaneous non-bursting, and values greater than 1 indicate more simultaneous bursting and/or simultaneous non-bursting than is expected by chance. To generate confidence limits on the value of concurrence, a bootstrap analysis was performed across trials.

Within each of the four brain regions examined (dlPFC, M1, CN and Put), the concurrence of bursts was statistically significantly greater than chance during ITI, but during at least the post-cue period and in some cases throughout the entire task execution period the concurrence of bursts was not statistically significantly greater than chance. In sum, the timing of beta bursts is substantially random during task performance, and become significantly more coherent (e.g., coordinated) across brain regions after task performance. Relative measurement of the timing of bursts therefore can be used to monitor task engagement in real time. In addition, burst rate can be used to monitor task engagement in real time.

Within each brain structure, each time point was marked where there was a greater-than-threshold number of simultaneous bursts in progress as belonging to a concurrent burst for that structure, and repeated the concurrence analysis on concurrent bursts across structures. As in the within-structure analysis, concurrence of concurrent bursts across structures is significantly greater than chance during ITI but not during post-Cue period. In addition, cross-structure concurrence was significantly greater during the entire ITI than at any time during the entire post-Cue and Movement period, and cross-structure concurrence showed a significant peak during the final hold period followed by a marginally significant valley during and shortly after the reward delivery period.

The cross-covariance of the envelopes of the composite HHT beta-band signals between CN and dlPFC also was analyzed using the same time periods as coherence. The majority of electrode pairs showed greater envelope cross-covariance when both electrodes were bursting than when both were non-bursting. This last result shows that the detailed fine temporal structure of beta bursts is similar across sites, further indicating the possibility that the sites are involved in concerted network-level interactions.

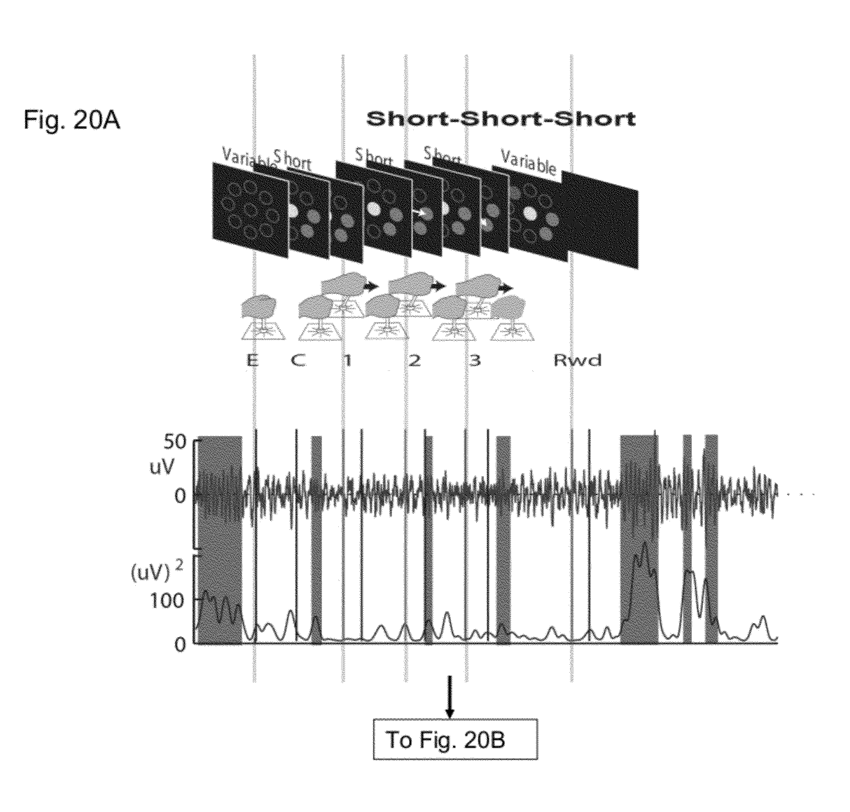

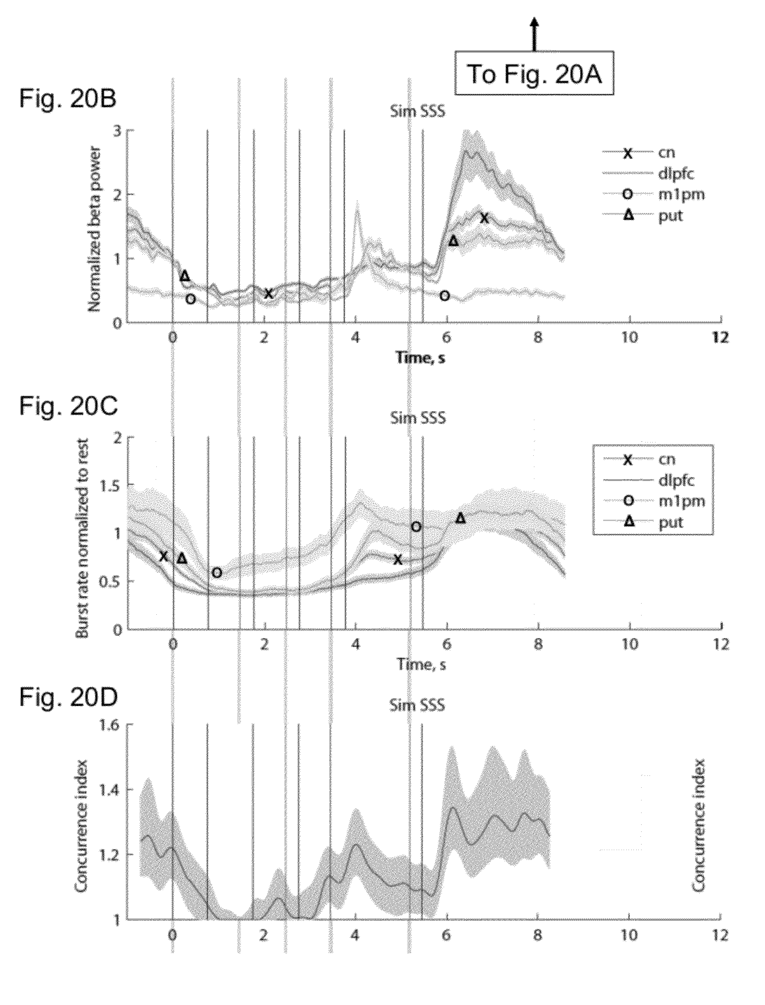

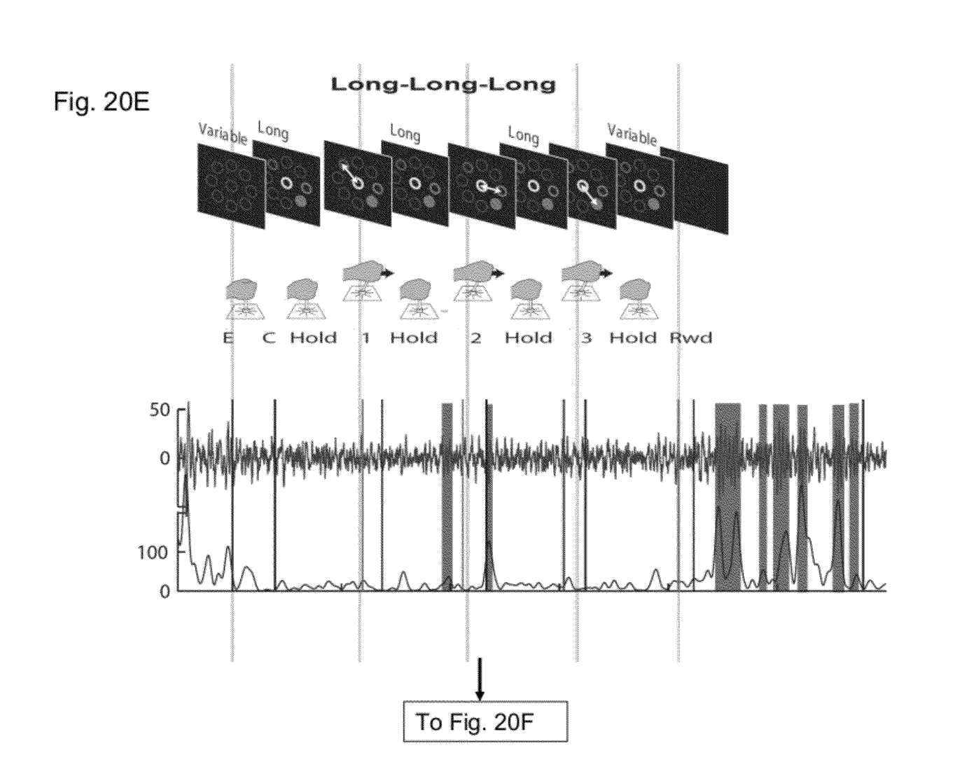

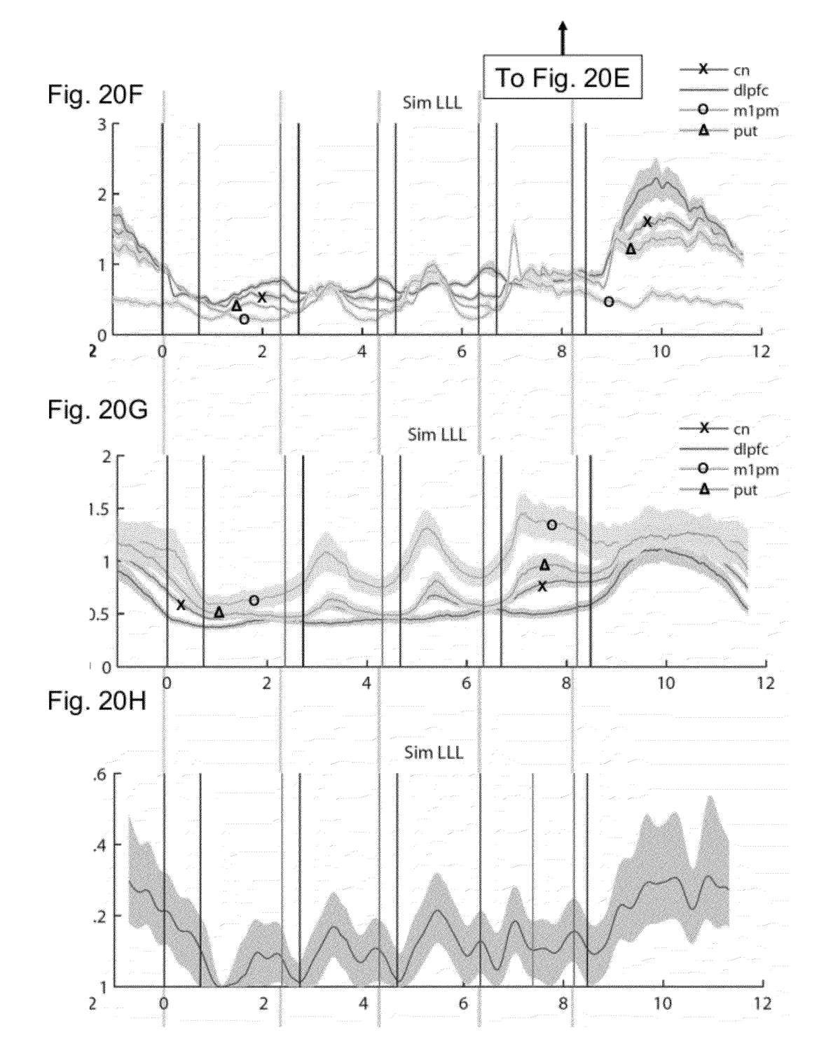

Referring to FIGS. 20A-H, a summary is shown of the various measures that can be used to detect an individual's (and individual brain region's) engagement in task performance, and the point at which the performance is (correctly) completed, namely: LFP power in the beta frequency range, beta burst rate, beta burst power, and the concurrence (or coordination) of beta bursts within and across brain structures.

The present invention may be embodied in many different forms, including, but in no way limited to, computer program logic for use with a processor (e.g., a microprocessor, microcontroller, digital signal processor, or general purpose computer), programmable logic for use with a programmable logic device, (e.g., a Field Programmable Gate Array (FPGA) or other PLD), discrete components, integrated circuitry (e.g., an Application Specific Integrated Circuit (ASIC)), or any other means including any combination thereof. In one embodiment of the present invention, some or all of the processing of the data collected is implemented as a set of computer program instructions that is converted into a computer executable form, stored as such in a computer readable medium, and executed by a microprocessor under the control of an operating system.

In some embodiments, computer software (e.g., a computer program) is provided for capturing, visualizing, and/or analyzing beta bursts.

The computer program may be fixed in any form (e.g., source code form, computer executable form, or an intermediate form) either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies networking technologies, and internetworking technologies. The computer program may be distributed in any form as a removable storage medium with accompanying printed or electronic documentation (e.g., shrink-wrapped software), preloaded with a computer system (e.g., on system ROM or fixed disk), or distributed over a network.

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink-wrapped software), preloaded with a computer system (e.g., on system ROM or fixed disk), or distributed from a server or electronic bulletin board over the communication system (e.g., the Internet or World Wide Web).

Computers and computer systems described herein may include operatively associated computer-readable media such as memory for storing software applications used in obtaining, processing, storing and/or communicating data. It can be appreciated that such memory can be internal, external, remote or local with respect to its operatively associated computer or computer system.

The aspects, embodiments, features, and examples of the invention are to be considered illustrative in all respects and are not intended to limit the invention, the scope of which is defined only by the claims. Other embodiments, modifications, and usages will be apparent to those skilled in the art without departing from the spirit and scope of the claimed invention.

The use of headings and sections in the application is not meant to limit the invention; each section can apply to any aspect, embodiment, or feature of the invention.

Throughout the application, where processes are described as having, including or comprising specific process steps, it is contemplated that processes also consist essentially of, or consist of, the recited process steps.

In the application, where an element or component is said to be included in and/or selected from a list of recited elements or components, it should be understood that the element or component can be any one of the recited elements or components and can be selected from a group consisting of two or more of the recited elements or components.

The use of the terms “include,” “includes,” “including,” “have,” “has,” or “having” should be generally understood as open-ended and non-limiting unless specifically stated otherwise.

The use of the singular herein includes the plural (and vice versa) unless specifically stated otherwise. Moreover, the singular forms “a,” “an,” and “the” include plural forms unless the context clearly dictates otherwise.

It should be understood that the order of steps or order for performing certain actions is immaterial so long as the invention remains operable. Moreover, two or more steps or actions may be conducted simultaneously.

What is claimed is:

1. A method of assessing whether an individual is engaged in a task, the method comprising the steps of:
measuring an EEG, for a plurality of frequencies, of the individual prior to, during and after task performance using a plurality of electrodes to generate a data set comprising frequencies and power;
selecting, using a computer, frequencies in the beta frequency range from the EEG data set;
comparing, using a computer, the power in the frequencies in the beta frequency range prior to, during and after task performance; and
determining whether the person was engaged in the task in response to the comparison of the power in the beta frequency range prior to, during, and after task performance.

2. The method of claim 1 wherein the comparison is made by plotting frequency and power against time.

3. The method of claim 1 wherein the measurement of task engagement is made for a plurality of brain regions.

4. The method of claim 3 further comprising the step of measuring coherence in the beta frequency range between brain regions of the plurality of brain regions.

5. The method of claim 1 wherein the comparison is performed by a computer by transforming the data.

6. The method of claim 5 wherein the transform is a filter.

7. The method of claim 5 wherein the transform is a HHT transform.

8. The method of claim 4 wherein the transform is an FFT transform.

9. The method of claim 1 wherein the individual is considered to have been engaged in the task if beta frequencies prior to task performance during resting have decreased power relative to the beta frequencies after task performance.

10. The method of claim 1, wherein the beta frequency range is about 13 Hz to about 30 Hz.

11. A method of assessing whether a brain region is engaged in a task, the method comprising: monitoring, using a computer, brain activity in the beta frequency range prior to, during, and after performance of a task, wherein a burst in beta frequency activity after performance of the task is indicative that the brain region has disengaged from performance of the task.

12. The method of claim 11, wherein the brain region is selected from the group consisting of primary motor and dorsal premotor cortex, dorsolateral prefrontal cortex, caudate nucleus, and putamen.

13. A method of diagnosing a condition affecting movement or thought preparation and movement cessation in an individual, the method comprising: measuring, using an electroencephalographic computer, brain activity in the beta frequency range prior to, during, and post performance of a task, wherein a burst in beta frequency activity during the task completion time in a normal brain region is indicative that the brain region has prematurely disengaged from performance of the task.

14. The method of claim 13, comprising measuring beta frequency activity in primary motor and dorsal premotor cortex, dorsolateral prefrontal cortex, caudate nucleus, and putamen.

15. A method of diagnosing a condition affecting movement or thought preparation and cessation in an individual, the method comprising: measuring, using a computer, brain activity in the beta frequency range before, during and after performance of a task, wherein bursts in beta frequency activity that are not coherent with bursts in other brain regions, as compared to normal brains, are indicative that brain region activity is abnormally coordinated.

16. The method of claim 15, comprising measuring the coherence of beta frequency activity in at least two brain regions.

17. The method of claim 15, wherein the at least two brain regions comprise striatum and prefrontal cortex.

18. The method of claim 15, wherein the at least two brain regions comprise caudate nucleus and dorsolateral prefrontal cortex.

19. An apparatus for assessing whether an individual is engaged in a task, the apparatus comprising:
a plurality of electrodes for placement on the head of the individual; and
a computer system comprising:
an EEG module, in communication with the plurality of electrodes, for measuring an EEG, for a plurality of frequencies, of the individual prior, during and post task using a plurality of electrodes to generate a data set of frequencies and power;
a selection module in communication with the EEG module for selecting frequencies in the beta frequency range from the EEG data set;
a comparator for comparing the power in the frequencies in the beta frequency range before, during and post task; and
a display, in electrical communication with the comparator for showing whether the person was engaged in the task, in response the comparison of the power in the beta frequency range before, during, and post task.

20. A method of assessing the efficacy of a drug used to treat an individual with a cognitive disability typified by hyperactive beta oscillations, the method comprising the steps of:
measuring an EEG at a first time, for a plurality of frequencies, of the individual prior to, during and after task performance using a plurality of electrodes to generate a first data set comprising frequencies and power, the first time being prior to administration of the drug;
measuring an EEG at a second time, for a plurality of frequencies, of the individual prior to, during and after task performance using a plurality of electrodes to generate a second data set comprising frequencies and power, the second time being after administration of the drug;
selecting, using a computer, frequencies in the beta frequency range from the first EEG data set and the second EEG data set;
comparing, using a computer, the power in the frequencies in the beta frequency range prior to, during and after task performance in the first EEG data set and in the second EEG data set; and
determining whether administration of the drug decreased beta frequency oscillations.

21. The method of claim 20, wherein cognitive disability is selected from the group consisting of: Parkinson's Disease, obsessive compulsive disorder, autism, attention deficit disorder, attention deficit hyperactivity disorder, and post traumatic stress disorder.

22. The method of claim 20, wherein the cognitive disability is Parkinson's Disease.

23. A method of assessing whether an individual is engaged in a task, the method comprising the steps of:
measuring an EEG, for a plurality of frequencies, of the individual prior to, during and after task performance using a plurality of electrodes to generate a data set comprising frequencies and burst rate;
selecting, using a computer, frequencies in the beta frequency range from the EEG data set;
comparing, using a computer, the burst rate in the frequencies in the beta frequency range prior to, during and after task performance; and
determining whether the person was engaged in the task in response to the comparison of the burst rate in the beta frequency range prior to, during, and after task performance.
